(19) United States
(12) Patent Application Publication
(10) Pub. No.: US 2025/0074876 A1
(43) Pub. Date: Mar. 6, 2025

(54) DIPHENOLIC COMPOUNDS AND PREPARATION METHODS THEREOF

(71) Applicant: PURPANA (BEIJING) TECHNOLOGIES CO., LTD., Beijing (CN)

(72) Inventors: Wenlong XING, Beijing (CN); Ti JIAO, Beijing (CN); Shengxue LI, Beijing (CN); Zhihui WANG, Beijing (CN)

(73) Assignee: PURPANA (BEIJING) TECHNOLOGIES CO., LTD., Beijing (CN)

(21) Appl. No.: 18/950,156

(22) Filed: Nov. 17, 2024

Related U.S. Application Data
(63) Continuation of application No. PCT/CN2023/085442, filed on Mar. 31, 2023.

(30) Foreign Application Priority Data
May 17, 2022 (CN) 202210534437.8
Nov. 24, 2022 (CN) 202211487425.0

(51) Int. Cl. C07D 211/76; A61K 8/34; A61K 8/49; A61K 31/05; A61K 31/085; A61K 31/4025; A61K 31/45; A61K 31/55; A61Q 19/10; C07C 39/15; C07C 43/253; C07D 207/27; C07D 223/10

(52) U.S. Cl. CPC C07D 211/76; A61K 8/347; A61K 8/4906; A61K 8/4913; A61K 8/4926; A61K 31/05; A61K 31/085; A61K 31/4025; A61K 31/45; A61K 31/55; A61Q 19/10; C07C 39/15; C07C 43/253; C07D 207/27; C07D 223/10; C07C 2601/16; C07C 2602/10

(57) ABSTRACT

A compound represented by a formula (A),

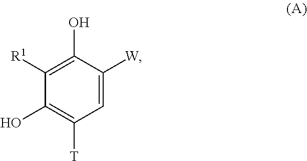

W is

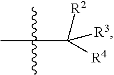

and T is H,

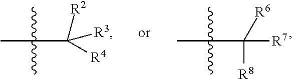

and R¹-R⁸ are defined as defined above.

### CROSS-REFERENCE TO RELATED APPLICATIONS

This application is a Continuation of International Application No. PCT/CN2023/085442, filed on Mar. 31, 2023, which claims priority to Chinese Patent Application No. 202210534437.8, filed on May 17, 2022, and to Chinese Patent Applications No. 202211487425.0, filed on Nov. 24, 2022, the entire contents of each of which are hereby incorporated by reference.

### TECHNICAL FIELD

The present disclosure relates to the field of diphenolic compounds, and in particular, to a diphenolic compound and a preparation method thereof.

### BACKGROUND

Melanin, which is a biological pigment responsible for skin coloration, is found in melanocytes. Melanin is generated by catalyzing tyrosine with tyrosinase. Melanin is overproduced due to excessive sun exposure, hormonal imbalances, melasma, diseases, medication injuries, scars, and age spots due to aging, which in turn causes hyperpigmentation of the skin.

The color of human skin is determined primarily by the amount of melanin in a basal layer of the skin. Melanin, which is usually brown to black in color, is formed in melanocytes and is transferred to stratum corneum of the skin to make the skin or hair brown or black. For mammals, brownish-black true melanin is formed primarily from aromatic amino acids substituted by hydroxyl, such as L-tyrosine and L-3,4-dihydroxyphenylalanine (L-DOPA). In addition, yellow to red pheomelanin is formed by sulfur-containing molecules. L-DOPA is formed by oxidizing L-tyrosine using tyrosinase, L-DOPA is converted to dopachrome using tyrosinase, and the dopachrome is finally oxidized to form melanin through a series of catalytic steps by a variety of enzymes.

When too much melanin is concentrated in one area or portion of the skin, the skin manifests as hyperpigmentation. Hyperpigmentation may also occur due to exposure to sunlight or different inflammatory stimuli. Hyperpigmentation usually accompanies with diseases such as blackspot, melasma or solar lentigines (senile plaques), freckles, and actinic keratosis. Blackspot is a general term describing dullness of the skin. Melasma is used to describe hormone-induced discoloration of the skin, and the hormone variation is usually caused by pregnancy, birth control pills, or estrogen-replacement therapy, etc. Solar lentigines are a dark discoloration of the skin caused by the sunlight, which are common on the skin of adults who are exposed to the sunlight for long periods of time. Although skin injuries, such as scars, wounds, or rashes, also lead to hyperpigmentation, the most common reason of skin darkening and brown spots is long periods exposure to sunlight.

At present, hyperpigmentation is mainly dealt with using brighteners. Common brighteners include hydroquinone and vitamin C, the mechanism of action of which is to brighten the skin by inhibiting the activity of tyrosinase to reduce the production of melanin. Common ingredients used for skin brightening include hydroquinone, arbutin, kojic acid, licorice extract, niacinamide, etc. Hydroquinone is one of the most effective decolorizers, but its use in cosmetics is limited due to its potential dermatological and systemic side effects. Arbutin is a glycosylated derivative of hydroquinone and a potent tyrosinase inhibitor, which is more stable and less toxic than hydroquinone, but arbutin is still unstable and susceptible to hydrolysis under different conditions.

Resorcinol compounds have great potential for inhibiting the activity of tyrosinase. Therefore, there is a need for developing a novel resorcinol compound, which is safer and more effective in treating or ameliorating hyperpigmentation of the skin.

### SUMMARY

Embodiments of the present disclosure provide a compound, which is represented by a formula (A),

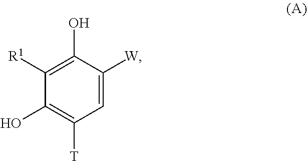

### W is

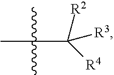

### and T is H,

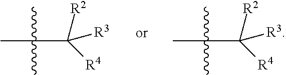

R¹ is selected from H, an optionally substituted alkyl group, and an optionally substituted arylalkyl group; R² and R⁶ are the same or different, which are independently selected from an optionally substituted aryl group, an optionally substituted heteroaryl group, the optionally substituted alkyl group, an optionally substituted amide group, and an optionally substituted ester group; R³, R⁴, R⁷, and R⁸ are the same or different, which are independently selected from H, the optionally substituted alkyl, an optionally substituted cycloalkyl group, and the optionally substituted amide group, wherein R³, R⁴, and a carbon atom to which R³ and R⁴ are both connected, are connected to form a ring, R⁷, R⁸, and a carbon atom to which R⁷ and R⁸ are commonly connected, are connected to form a ring, wherein the optionally substituted amide group is preferably —NHCOCH₃, —NHCOH, —NHCOCH₂CH₃, —NHCOCH₂CH₂CH₃, —NHCOCH(CH₃)₂, —NHCOCH(CH₂)₂, —N(COCH₃)₂, —CONH₂, —CON(CH₃)₂, —CONHCH₃, —CONHCH₂CH₃, —CON(CH₂CH₃)₂, —CONHCH(CH₃)₂, —CONHCH₂CH₂CH₃, —CONHCH₂CH₂CH₂CH₃, a phthalimido group, a succinimidyl group, a glutarimido group, a maleimido group,

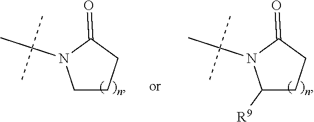

R⁹ is selected from the optionally substituted alkyl group, the optionally substituted aryl group, a carboxyl or a salt thereof, a cyano group, the optionally substituted amide group, and the optionally substituted ester group, n is a natural number from 1 to 6, and the optionally substituted ester group is —COOCH₃, —COOCH₂CH₃, —COOCH(CH₃)₂, —COOCH₂CH₂CH₃, or —COOCH₂CH₂CH₂CH₃; or a cosmetic acceptable salt thereof or a pharmaceutically acceptable salt thereof, stereoisomers or mixtures of the stereoisomers, enantiomers or mixtures of the enantiomers, or racemic mixtures.

In some embodiments, the compound is a compound represented by a formula (I),

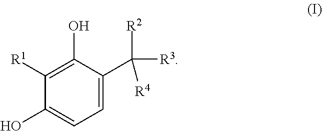

In some embodiments, the compound is a compound represented by a formula (Ia),

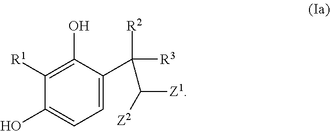

Z¹ and Z² are the same or different, which are independently selected from H, the optionally substituted alkyl group, the optionally substituted cycloalkyl group, and the optionally substituted amide group, Z¹, Z², and a carbon atom to which Z¹ and Z² are commonly connected, are connected to form a ring, and the ring is substituted.

In some embodiments, the compound is a compound represented by a formula (V),

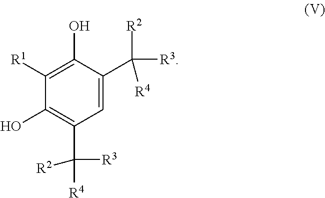

In some embodiments, the compound is a compound represented by a formula (Va),

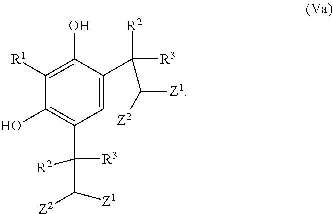

Z¹ and Z² are the same or different, which are independently selected from H, the optionally substituted alkyl group, the optionally substituted cycloalkyl group, and the optionally substituted amide group, the Z¹, Z², and a carbon atom to which Z¹ and Z² are commonly connected, are connected to form a ring, and the ring is substituted.

In some embodiments, the compound is a compound represented by a formula (VI),

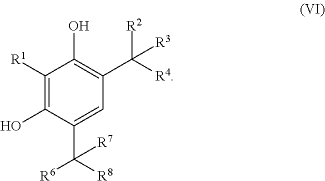

In some embodiments, the compound is a compound represented by a formula (VIa).

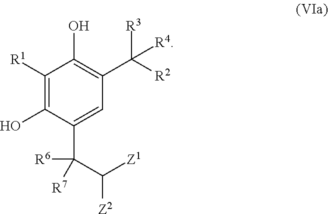

Z¹ and Z² are the same or different, which are independently selected from H, the optionally substituted alkyl group, the optionally substituted cycloalkyl group, and the optionally substituted amide group, Z¹, Z², and a carbon atom to which Z¹ and Z² are commonly connected, are connected to form a ring, and the ring is substituted.

Embodiments of the present disclosure also provide a method for preparing the compound represented by the formula (A).

Embodiments of the present disclosure also provide a pharmaceutical composition including the compound represented by the formula (A) or a pharmaceutically acceptable salt thereof.

Embodiments of the present disclosure also provide a cosmetic composition, including the compound represented by the formula (A) or a pharmaceutically acceptable salt thereof.

### DETAILED DESCRIPTION

In order to make the purpose, technical solutions and advantages of the embodiments of the present disclosure clear, the technical solutions in the embodiments of the present disclosure will be described clearly and completely in the following. It is clear that the described embodiments are a part of the embodiments of the present disclosure, and not all of the embodiments. Based on the embodiments in the present disclosure, all other embodiments obtained by those ordinary skilled in the art without creative labor fall within the scope of protection of the present disclosure. Unless otherwise expressly indicated, the terms “a,” “one,” “a kind of,” and/or “the” throughout the present disclosure and claims do not refer specifically to the singular, but may also include the plural. The term “including” or its transformations such as “contains” or “includes”, etc. will be understood to include the stated composition or step and not exclude other material compositions or steps.

Neither the endpoints of the ranges and any values disclosed herein are limited to that precise range or value, and those ranges or values should be understood to include values close to those ranges or values. For numerical ranges, the range between endpoint values, the endpoint values of the ranges, and the individual point values may be combined with each other to obtain one or more new numerical ranges, and these numerical ranges shall be deemed to be specifically disclosed herein.

As used herein, the term “hyperpigmentation” refers to excessive pigmentation of the skin caused by sunlight or visible light.

As used herein, the term “cosmetically or pharmaceutically acceptable” refers to be used in the preparation of a cosmetic or pharmaceutical composition that is generally non-toxic, safe, and acceptable for pharmaceutical and cosmetic use.

As used herein, the term “cosmetically or pharmaceutically acceptable salt” refers to a cosmetically or pharmaceutically acceptable salt having properties and activity of the original compound. The cosmetically or pharmaceutically acceptable salt may include: (a) an acid addition salt formed by the original compound with inorganic acid, and the inorganic acid includes but is not limited to hydrochloric acid, hydrobromic acid, sulfuric acid, nitric acid, phosphoric acid, etc.; or an acid addition salt formed by the original compound with organic acid, and the organic acid includes but is not limited to acetic acid, benzenesulfonic acid, benzoic acid, camphorsulfonic acid, citric acid, ethylsulfonic acid, fumaric acid, glucoheptonic acid, gluconic acid, glutamic acid, glycolic acid, hydroxynaphthylacetic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid, etc.; and (b) a salt formed when an acid proton in the original compound is substituted with a metal ion or an aluminum ion, the metal ion including an alkali metal ion (such as Na⁺, K⁺, or Li⁺), an alkaline-earth metal ion (such as Ca²⁺ or Mg²⁺), etc.; or a salt formed when the acid proton in the original compound is coordinated to an organic base or an inorganic base. An acceptable organic base includes, but is not limited to, diethanolamine, ethanolamine, N-methylglucosamine, triethanolamine, trometamol, etc. An acceptable inorganic base includes, but is not limited to, aluminum hydroxide, calcium hydroxide, potassium hydroxide, sodium carbonate, and sodium hydroxide, etc.

As used herein, the term “stereoisomer” refers to configurational stereoisomers, which includes geometric isomers, optical isomers, and conformational isomers.

Geometric isomers (also known as E/Z isomers or cis-trans isomers) are caused by different positions of the substituent groups on the C═C double bond. Geometric isomers include Z or E configuration, also known as the cis or trans configuration.

Optical isomers are caused by different positions of substituents or lone pair electrons of an atom (e.g., carbon or sulfur atoms) including four different substituents (potentially including lone-pair electrons) in space. Thus, the atom has a chiral center or an asymmetric center. Optical isomers that are not mirror images of each other are therefore called “diastereoisomers”, while optical isomers that are non-overlapping mirror images of each other are called “enantiomers”.

An equimolar mixture of two enantiomers of a chiral compound is called a racemic mixture.

As used herein, the term “halogen” in all cases denotes fluorine, chlorine, bromine, and iodine.

The term “alkyl group” as used herein (as well as other groups including an alkyl group, e.g., the alkyl portion of an alkoxy group, the alkyl portion of an arylalkyl group) generally denotes, in each case, a straight or branched chain alkyl group having 1-20 carbon atoms, 1-6 carbon atoms, 1-4 carbon atoms, or 1-3 carbon atoms. Examples of C₁-C₄ alkyl groups are methyl, ethyl, n-propyl, isopropyl, n-butyl, 1-methylpropyl (sec-butyl), 2-methylpropyl (isobutyl), 1,1-dimethylethyl (tert-butyl). Examples of C₁-C₆ alkyl groups, in addition to those mentioned for C₁-C₄ alkyl groups, include but are not limited to n-pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl. Examples of C₁-C₁₀ alkyl groups, in addition to those mentioned for C₁-C₆ alkyl groups, include, but are not limited to, n-heptyl, 1-methylhexyl, 2-methylhexyl, 3-methylhexyl, 1-ethylhexyl, 2-ethylhexyl, 1,2-dimethylhexyl, 1-propylpentyl, 2-propylpentyl, nonyl, decyl, 2-propylheptyl, and 3-propylheptyl. The alkyl group may be, but is not limited to, substituted with a halogen, a cyano group, a nitro group, an aryl group, a cycloalkyl group, a heterocyclic group, an amide group, an ester group, or the like.

As used herein, the term “aryl group” refers to a monovalent monocyclic or bicyclic aromatic hydrocarbon group of 6 to 10 carbon atoms, such as, but not limited to, a phenyl group or a naphthyl group. The aryl group may be, but is not limited to, substituted with an alkyl group, a halogen, a cyano group, a nitro group, a cycloalkyl group, a heterocyclic group, an amide group, an ester group, or the like.

As used herein, the term “ring” refers to “a cycloalkyl group” or “a heterocyclic group”, and the “cycloalkyl group” refers to a monocyclic monovalent hydrocarbon group of 3 to 6 carbon atoms, which is saturated or contains a double bond. The “cycloalkyl group” may be unsubstituted or substituted with one or two substituents independently selected from an alkyl group, a halogen, an alkoxy group, a hydroxyl group, or a cyano group, but not limited thereto. Examples of the “cycloalkyl group” include, but are not limited to, cyclopropyl, cyclobutyl, cyclopentyl, cyclohexyl, 1-cyanocyclopropan-1-yl, 1-cyanomethylcyclopropan-1-yl, 3-fluorocyclohexyl, and the like. When the cycloalkyl group contains a double bond, it may be referred to herein as a cycloalkenyl group. The “heterocyclic group” refers to a saturated or unsaturated monovalent monocyclic group of 4 to 8 ring atoms, one or two of the ring atoms are heteroatoms selected from N, O, or S(O)ₚ, p is an integer from 0 to 2, and the remaining ring atoms is C. In addition, one or two carbon atoms in the ring of the heterocyclic group may optionally be replaced by —CO— group. Specifically, the term “heterocyclic group” includes, but is not limited to, azetidinyl, oxetidinyl, pyrrolidinyl, piperidinyl, homopiperidinyl, 2-oxopyrrolidinyl, 2-oxopiperidinyl, morpholino, piperazino, tetrahydropyranyl, thiomorpholino, or the like. The cycloalkyl group and the heterocyclic group may be, but are not limited to, substituted with an alkyl group, a halogen, a cyano group, a nitro group, an aryl group, an amide group, an ester group, or the like.

The term “optionally substituted” means that a relevant group may or may not be substituted.

When a group is substituted, a substituent includes, but is not limited to, an alkyl group, a halogen, a cyano group, a nitro group, a cycloalkyl group, a heterocyclic group, an amide group, an ester group, and an alkoxy group, etc., for example, when an alkyl group is substituted by a halogen, an alkyl halide is formed

The term “alkoxy group” refers to an —OR group, R being an alkyl group as defined above, e.g., methoxy, ethoxy, propoxy, 2-propoxy, n-butoxy, isobutoxy, or tert-butoxy, or the like.

As used herein, the term “amide group” refers to a group containing “—NHCO—” or

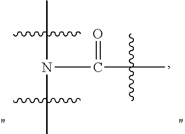 [SMILES: *C(=O)N(*)*]

e.g., —NHCOCH₃, —NHCOH, —NHCOCH₂CH₃, —NHCOCH₂CH₂CH₃, —NHCOCH(CH₃)₂, —NHCOCH(CH₂)₂, —NHCOCH₂CH₃, —NHCOCH₂CH₂CH₃, —NHCOCH(CH₃)₂, —NHCOCH(CH₂)₂, —N(COCH₃)₂, —CONH₂, —CON(CH₃)₂, —CONHCH₃, —CONHCH₂CH₃, —CON(CH₂CH₃)₂, —CONHCH(CH₃)₂, —CONHCH₂CH₂CH₃, —CONHCH₂CH₂CH₂CH₃, a phthalimido group, a succinimidyl group, a glutarimido group, a maleimido group,

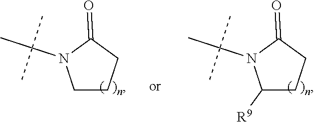

etc., R⁹ is selected from an optionally substituted alkyl group, an optionally substituted aryl group, a carboxyl group or a salt thereof, a cyano group, an optionally substituted amide group, and an optionally substituted ester group, n is a natural number in 1-6, and the ester group includes but not limited to —COOCH₃, —COOCH₂CH₃, —COOCH(CH₃)₂, —COOCH₂CH₂CH₃, and —COOCH₂CH₂CH₂CH₃.

As used herein, the term “ester group” refers to a group containing “—COO—”, e.g., —COOCH₃, —COOCH₂CH₃, —COOCH(CH₃)₂, —COOCH₂CH₂CH₃, —COOCH₂CH₂CH₂CH₃, or the like.

As used herein, the term “heteroaryl group” includes an aromatic monocyclic ring with 5-10 members and an aromatic thick ring with 5-10 members. The aromatic monocyclic ring contains one or more (e.g., 1 to 4, or 1 to 3) heteroatoms selected from N, O, and S and the remaining atoms are carbon atoms. The aromatic condensed ring contains one or more (e.g., 1 to 4, or 1 to 3) heteroatoms selected from N, O, and S and the remaining atoms are carbon atoms and at least one of the heteroatoms is present in the aromatic ring. For example, the heteroaryl group includes a heterocycloalkyl aryl ring with 5-10 members that is condensed by a cycloalkyl group or a heterocycloalkyl ring with 5-10 members. For a heteroaryl bicyclic ring in which only one ring contains one or more heteroatoms, the point of attachment may be on any ring. When the total number of S and O atoms in the heteroaryl group exceeds 1, the heteroatoms are not adjacent to each other. In some embodiments, the total number of S and O atoms in the heteroaryl group does not exceed 2. In some embodiments, the total number of S and O atoms in the heteroaryl group does not exceed 1. In some embodiments, the heteroaryl group includes, but is not limited to, 2-pyridinyl, 3-pyridinyl, 4-pyridinyl, 2,3-pyridazinyl, 3,4-pyridazinyl, 2,4-pyrimidinyl, 3,5-pyrimidinyl, 2,3-pyrazolyl, 2,4-imidazolyl, isoxazolyl, oxazolyl, thiazolinyl, thiadiazolinyl, tetrazolyl, thiophenyl, benzothiophenyl, furanyl, benzofuranyl, benzimidazolyl, indolinyl, pyrazinyl, triazolyl, quinolinyl, pyrazolyl and 5,6,7,8-tetrahydroisoquinolinyl. The heteroaryl group does not include aryl group, cycloalkyl group, or heterocycloalkyl group, or does not overlap with an aryl group, a cycloalkyl group, or a heterocycloalkyl group, as defined herein.

As used herein, the term “arylalkyl group” refers to a residue in which an aryl portion is connected to the parent structure by an alkyl residue. Examples of the arylalkyl group include benzyl, phenylethyl, phenyl vinyl, phenyl allyl, or the like. The term “heteroarylalkyl group” refers to a residue in which a heteroaryl portion is connected to the parent structure by an alkyl residue. Examples of the heteroarylalkyl group include furylmethyl, pyridinylmethyl, pyrimidinylethyl, or the like.

The following descriptions of the variable and variant embodiments of a compound represented by a formula (A), the characterization of the methods of the present disclosure, and the characterization of compositions of the present disclosure are valid either by themselves or preferably in combination with each other.

Embodiments of the present disclosure provide a resorcinol compound, which is represented by the formula (A):

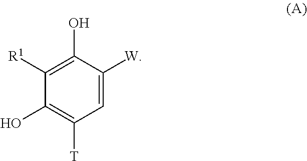

W is

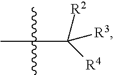

### and T is H,

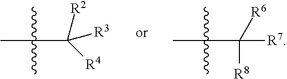

R¹ is selected from H, an optionally substituted alkyl group, and an optionally substituted arylalkyl group.

R² and R⁶ are the same or different, which are independently selected from an optionally substituted aryl group, an optionally substituted heteroaryl group, the optionally substituted alkyl group, an optionally substituted amide group, and an optionally substituted ester group; R³, R⁴, R⁷ and R⁸ are the same or different, which are independently selected from H, the optionally substituted alkyl group, an optionally substituted cycloalkyl group, and the optionally substituted amide group, R³, R⁴, and a carbon atom to which R³ and R⁴ are both connected, are connected to form a ring, and R⁷, R⁸, and a carbon atom to which R⁷ and R⁸ are commonly connected, are connected to form a ring. The optionally substituted amide group is —NHCOCH₃, —NHCOH, —NHCOCH₂CH₃, —NHCOCH₂CH₂CH₃, —NHCOCH(CH₃)₂, —NHCOCH(CH₂)₂, —N(COCH₃)₂, —CONH₂, —CON(CH₃)₂, —CONHCH₃, —CONHCH₂CH₃, —CON(CH₂CH₃)₂, —CONHCH(CH₃)₂, —CONHCH₂CH₂CH₃, —CONHCH₂CH₂CH₂CH₃, a phthalimido group, a succinimidyl group, a glutarimido group, a maleimido group,

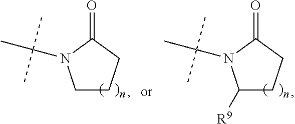

R⁹ is selected from the optionally substituted alkyl group, the optionally substituted aryl group, a carboxyl group or a salt thereof, a cyano group, the optionally substituted amide group, and the optionally substituted ester group, n is a natural number from 1 to 6, and the optionally substituted ester group is —COOCH₃, —COOCH₂CH₃, —COOCH(CH₃)₂, —COOCH₂CH₂CH₃, or —COOCH₂CH₂CH₂CH₃.

The resorcinol compound provided by embodiments of the present disclosure may be a cosmetically acceptable salt or a pharmaceutically acceptable salt of the compound represented by the formula (A), stereoisomers or mixtures of stereoisomers in any ratio, enantiomers or mixtures of enantiomers, or racemic mixtures.

In some embodiments, the compound is a compound represented by a formula (I) or a salt thereof:

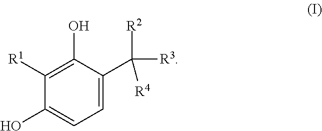

R¹ is selected from H, the optionally substituted alkyl group, and the optionally substituted arylalkyl group.

R² is selected from an optionally substituted aryl group, an optionally substituted alkyl group, an optionally substituted amide group, an optionally substituted ester group, and an optionally substituted heteroaryl group with 5-10 members (the heteroaryl group including 1 to 4 heteroatoms independently selected from nitrogen, oxygen, or sulfur), preferably the optionally substituted alkyl,

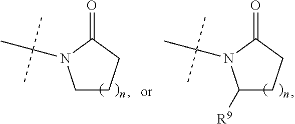

R⁹ is selected from the optionally substituted alkyl group or the optionally substituted aryl group, a carboxyl group or a salt thereof, a cyano group, an optionally substituted amide group, and an optionally substituted ester group, and n is a natural number from 1 to 6, for example, 1, 2, 3. R³ and R⁴ may be the same or different, which are independently selected from H, the optionally substituted alkyl, an optionally substituted cycloalkyl group, and the optionally substituted amide group, and R³, R⁴, and a carbon atom to which R³ and R⁴ are both connected, are connected to form a ring.

In some embodiments, R¹ is selected from H and C₁-C₆ alkyl group. In some embodiments, R¹ is selected from H and a methyl group.

In some embodiments, R² is

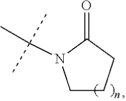

n is 1, 2, or 3.

In some embodiments, R² is

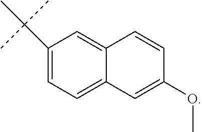

In some embodiments, R² is a phenyl group.

In some embodiments, R³ is selected from H and C₁-C₆ alkyl group. In some embodiments, R³ is selected from at least one of H, a methyl group, an ethyl group, a propyl group, and a butyl group.

In some embodiments, R⁴ is selected from H and C₁-C₆ alkyl group. In some embodiments, R⁴ is selected from at least one of H, a methyl group, an ethyl group, a propyl group, and a butyl group.

In some embodiments, the compound represented by the formula (I) includes compounds represented by formulas (I-1), (I-2), and (I-3):

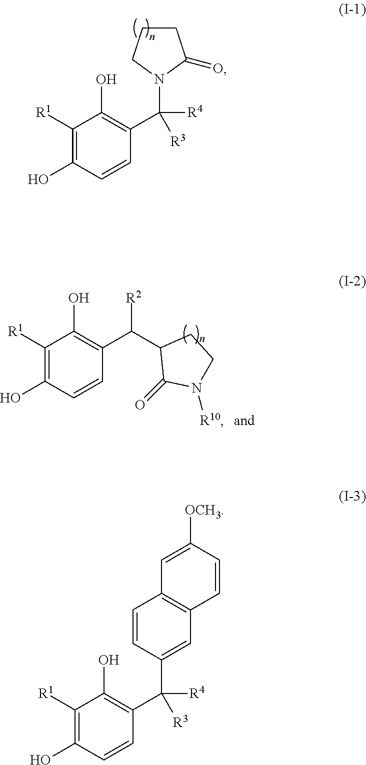

R¹ is selected from H and C₁-C₆ alkyl group. In some embodiments, R¹ is selected from H and the methyl group and R² is selected from a phenyl group. In some embodiments, R³ is selected from H and C₁-C₆ alkyl group. In some embodiments, R³ is selected from at least one of H, the methyl group, the ethyl group, the propyl group, and the butyl group, and R⁴ is selected from H and C₁-C₆ alkyl group. In some embodiments, R⁴ is selected from at least one of H, the methyl group, the ethyl group, the propyl group and the butyl group, and R¹⁰ is selected from C₁-C₁₈ alkyl group. In some embodiments, R¹⁰ is selected from an n-dodecyl group, an n-tetradecyl group, and an n-hexadecyl group and n is selected from 1, 2, and 3.

In some embodiments, the compound is a compound represented by a formula (Ia):

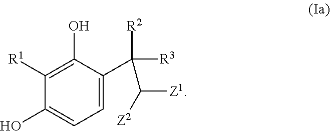

R¹ is selected from H, an optionally substituted alkyl group, and an optionally substituted arylalkyl group; R² is selected from an optionally substituted aryl group, an optionally substituted heteroaryl group with 5-10 members including 1-4 heteroatoms independently selected from nitrogen, oxygen, or sulfur, the optionally substituted alkyl group, and an optionally substituted amide group; and R³ is selected from H, the optionally substituted alkyl group, an optionally substituted cycloalkyl group, and the optionally substituted amide group; Z¹ and Z² are the same or different, which are independently selected from H, the optionally substituted alkyl group, the optionally substituted cycloalkyl group, and the optionally substituted amide group, Z¹, Z², and a carbon atom to which Z¹ and Z² are commonly connected, are connected to form a ring, and the ring is substituted.

In some embodiments, R¹ is selected from at least one of H and C₁-C₆ alkyl group. In some embodiments, in the compound represented by the formula (Ia), R¹ is selected from at least one of H and a methyl group.

In some embodiments, R² is selected from at least one of

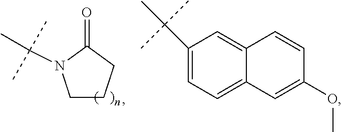

and a phenyl group, and n is a natural number from 1-6.

In some embodiments, the compound represented by the formula (Ia) includes compounds represented by formulas (Ia-1) and (Ia-2):

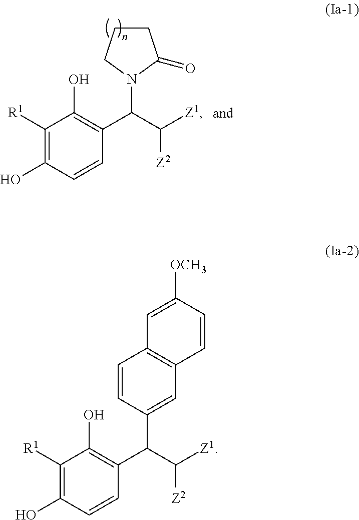

R¹ is selected from H and C₁-C₆ alkyl group. In some embodiments, R¹ is selected from at least one of H and the methyl group.

Z¹ and Z² are independently selected from H and C₁-C₆ alkyl group. In some embodiments, Z¹ and Z² are independently selected from at least one of H, a methyl group, an ethyl group, a propyl group, and a butyl group; or Z¹, Z², and a carbon atom to which Z¹ and Z² are commonly connected, form C₃-C₆ cycloalkyl group or 3-6 membered heterocyclic group. The C₃-C₆ cycloalkyl group includes cyclopropyl, cyclobutyl, cyclopentyl, cyclohexyl. The 3-6 membered heterocyclic group includes ethylene oxide, azetidinyl, oxetidinyl, pyrrolidinyl, piperidinyl, homopiperidinyl, 2-oxopyrrolidinyl, 2-oxopiperidinyl, morpholino, piperazinyl, tetrahydropyranyl, and thiomorpholinyl; and n is selected from a natural number from 1 to 6. In some embodiments, n is selected from 1, 2, and 3.

In some embodiments, the compound is a compound represented by a formula (V):

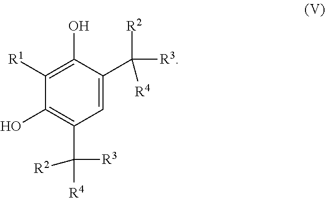

R¹ is selected from H, an optionally substituted alkyl group, and an optionally substituted arylalkyl group

R² is selected from an optionally substituted aryl group, the optionally substituted alkyl group, an optionally substituted amide group, and the optionally substituted amide group includes at least one of —NHCOCH₃, —NHCOH, —NHCOCH₂CH₃, —NHCOCH₂CH₂CH₃, —NHCOCH(CH₃)₂, —NHCOCH(CH₂)₂, —N(COCH₃)₂, —CONH₂, —CON(CH₃)₂, —CONHCH₃, —CONHCH₂CH₃, —CON(CH₂CH₃)₂, —CONHCH(CH₃)₂, —CONHCH₂CH₂CH₃, —CONHCH₂CH₂CH₂CH₃, —CONHCH₂CH₂CH₂CH₃, a phthalimido group, a succinimidyl group, a glutarimido group, a maleimido group,

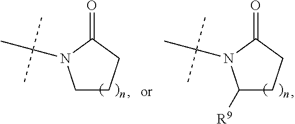

R⁹ is selected from the optionally substituted alkyl group, the optionally substituted aryl group, a carboxyl group or a salt thereof, an optionally substituted ester group, a cyano group, and the optionally substituted amide group, the optionally substituted ester group includes at least one of —COOCH₃, —COOCH₂CH₃, —COOCH(CH₃)₂, —COOCH₂CH₂CH₃, —COOCH₂CH₂CH₂CH₃, —COOCH₂CH₂CH₂CH₃, and n is selected from a natural number of 1 to 6. R³ and R⁴ are the same or different, which are independently selected from H, the optionally substituted alkyl group, an optionally substituted cycloalkyl group, and the optionally substituted amide group, R³, R⁴, and a carbon atom to which R³ and R⁴ are both connected, are connected to form a ring.

In some embodiments, the compound represented by the formula (V) is a compound represented by a formula (Va):

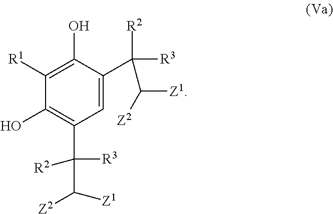

R¹ is selected from H, the optionally substituted alkyl group, and the optionally substituted arylalkyl group.

R² is selected from the optionally substituted aryl group, the optionally substituted alkyl group, the optionally substituted amide group, and the optionally substituted amide group includes at least one of —NHCOCH₃, —NHCOH, —NHCOCH₂CH₃, —NHCOCH₂CH₂CH₃, —NHCOCH(CH₃)₂, —NHCOCH(CH₂)₂, —N(COCH₃)₂, —CONH₂, —CON(CH₃)₂, —CONHCH₃, —CONHCH₂CH₃, —CON(CH₂CH₃)₂, —CONHCH(CH₃)₂, —CONHCH₂CH₂CH₃, —CONHCH₂CH₂CH₂CH₃, —CONHCH₂CH₂CH₂CH₃, a phthalimido group, a succinimidyl group, a glutarimido group, a maleimido group,

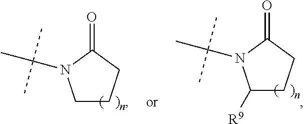

R⁹ is selected from the optionally substituted alkyl group, the optionally substituted aryl group, the carboxyl group or the salt thereof, the optionally substituted ester group, the cyano group, and the optionally substituted amide group, the ester group includes at least one of —COOCH₃, —COOCH₂CH₃, —COOCH(CH₃)₂, —COOCH₂CH₂CH₃, and —COOCH₂CH₂CH₂CH₃, and n is selected from a natural number from 1 to 6. R³ is selected from H, the optionally substituted alkyl group, the optionally substituted cycloalkyl group, and the optionally substituted amide group; Z¹ and Z² are the same or different, which are independently selected from H, the optionally substituted alkyl group, the optionally substituted cycloalkyl group, and the optionally substituted amide group, and Z¹, Z², and a carbon atom to which Z¹ and Z² are commonly connected, are connected to form a ring, and the ring is substituted.

In some embodiments, the compound is a compound represented by a formula (VI):

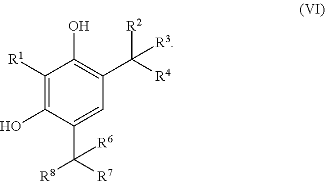

R¹ is selected from H, the optionally substituted alkyl group, and the optionally substituted arylalkyl group; R² and R⁶ are the same or different, which are independently selected from the optionally substituted aryl group, 5-10 membered heteroaryl group including 1 to 4 heteroatoms independently selected from nitrogen, oxygen, or sulfur, the optionally substituted alkyl group, the optionally substituted ester group, and the optionally substituted amide group, the optionally substituted amide group includes at least one of —NHCOCH₃, —NHCOH, —NHCOCH₂CH₃, —NHCOCH₂CH₂CH₃, —NHCOCH(CH₃)₂, —NHCOCH(CH₂)₂, —N(COCH₃)₂, —CONH₂, —CON(CH₃)₂, —CONHCH₃, —CONHCH₂CH₃, —CON(CH₂CH₃)₂, —CONHCH(CH₃)₂, —CONHCH₂CH₂CH₃, —CONHCH₂CH₂CH₂CH₃, the phthalimido group, the succinimidyl group, the glutarimido group, the maleimido group,

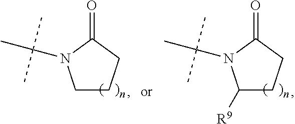

R⁹ is selected from the optionally substituted alkyl group, the optionally substituted aryl group, the carboxyl group or the salt thereof, the optionally substituted ester group, the cyano group, and the optionally substituted amide group, the optionally substituted ester group includes at least one of —COOCH₃, —COOCH₂CH₃, —COOCH(CH₃)₂, —COOCH₂CH₂CH₃, —COOCH₂CH₂CH₂CH₃, and n is selected from a natural number of 1-6. R³, R⁴, R⁷ and R⁸ are the same or different, which are independently selected from H, the optionally substituted alkyl group, the optionally substituted cycloalkyl group, and the optionally substituted amide group, R³, R⁴, and a carbon atom to which R³ and R⁴ are both connected, are connected to form a ring, and R⁷, R⁸, and a carbon atom to which R⁷ and R⁸ are commonly connected, are connected to form a ring, and the rings are substituted.

In some embodiments, R² and R⁶ are

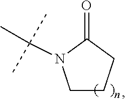

and n is a natural number from 1-6.

In some embodiments, R² and R⁶ are a phenyl group.

In some embodiments, the compound represented by the formula (VI) includes compounds represented by formulas (VI-1) and (VI-2):

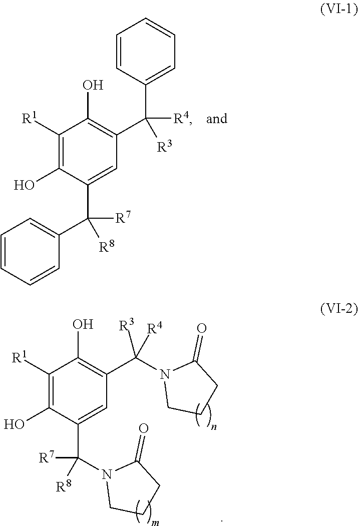

R¹ is selected from H and C₁-C₆ alkyl group. In some embodiments, R¹ is selected from at least one of H and a methyl group. R³ is selected from H and C₁-C₆ alkyl group. In some embodiments, R³ is selected from at least one of H, a methyl group, an ethyl group, a propyl group, and a butyl group. R⁴ is selected from H and C₁-C₆ alkyl group. In some embodiments, R⁴ is selected from at least one of H, a methyl group, an ethyl group, a propyl group, and a butyl group. R⁷ is selected from H and C₁-C₆ alkyl group. In some embodiments, R⁷ is selected from at least one of H, a methyl group, an ethyl group, a propyl group, and a butyl group. R⁸ is selected from H and C₁-C₆ alkyl group. In some embodiments, R⁸ is selected from at least one of a methyl group, an ethyl group, a propyl group, and a butyl group. n is selected from 1, 2, and 3, and m is selected from 1, 2, and 3.

In some embodiments, the compound represented by the formula (VI) is a compound represented by formula (VIa):

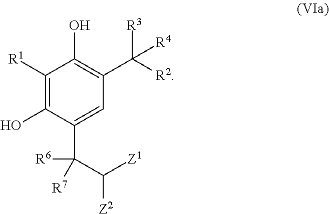

R¹ is selected from H, the optionally substituted alkyl group, and the optionally substituted arylalkyl group. R² and R⁶ are the same or different, which are independently selected from the optionally substituted aryl group, 5-10 membered heteroaryl group including 1 to 4 heteroatoms independently selected from nitrogen, oxygen, or sulfur, the optionally substituted alkyl group, the optionally substituted ester group, the optionally substituted amide group, the optionally substituted amide group includes at least one of —NHCOCH₃, —NHCOH, —NHCOCH₂CH₃, —NHCOCH₂CH₂CH₃, —NHCOCH(CH₃)₂, —NHCOCH(CH₂)₂, —N(COCH₃)₂, —CONH₂, —CON(CH₃)₂, —CONHCH₃, —CONHCH₂CH₃, —CON(CH₂CH₃)₂, —CONHCH(CH₃)₂, —CONHCH₂CH₂CH₃, —CONHCH₂CH₂CH₂CH₃, the phthalimido group, the succinimidyl group, the glutarimido group, the maleimido group,

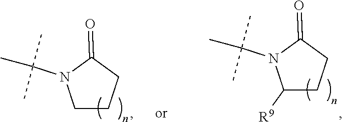

R⁹ is selected from the optionally substituted alkyl group, the optionally substituted aryl group, the carboxyl group or the salt thereof, the optionally substituted ester group, the cyano group, and the optionally substituted amide group, the optionally substituted ester group includes at least one of —COOCH₃, —COOCH₂CH₃, —COOCH(CH₃)₂, —COOCH₂CH₂CH₃, —COOCH₂CH₂CH₂CH₃, and n is selected from a natural number of 1-6. R³, R⁴, and R⁷ are the same or different, which are independently selected from H, the optionally substituted alkyl group, the optionally substituted cycloalkyl group, and the optionally substituted amide group, R³, R⁴, and a carbon atom to which R³ and R⁴ are both connected, are connected to form a ring. Z¹ and Z² are the same or different, which are independently selected from hydrogen, the optionally substituted alkyl group, the optionally substituted cycloalkyl group, the optionally substituted amide group, Z¹, Z², and a carbon atom to which Z¹ and Z² are commonly connected, are connected to form a ring, and the ring is substituted.

In some embodiments, the resorcinol compound includes the following compounds:

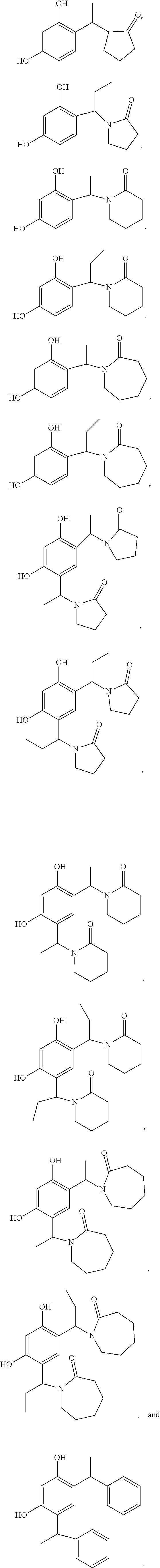

Embodiments of the present disclosure also provide a method for preparing the compound represented by the formula (I), the compound is prepared by reacting a compound represented by a formula (II) with a compound represented by a formula (IVa), and a preparation route (S1) is as follows:

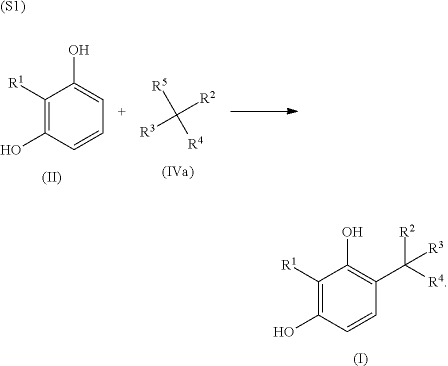

R¹, R², R³, and R⁴ are defined as defined above, and R⁵ is selected from a leaving group which is selected from at least one of OH, H₂O, OTs, OMs, Cl, Br, and I.

In some embodiments, a reaction temperature of the preparation route (S1) is within a range of 40-110° C. In some embodiments, the reaction temperature of the preparation route (S1) is within a range of 70-90° C.

In some embodiments, in the preparation route (S1), a molar ratio of the compound represented by the formula (II) to the compound represented by the formula (IVa) is 1:(1-1.5). In some embodiments, the molar ratio of the compound represented by the formula (II) to the compound represented by the formula (IVa) is 1:(1.01-1.05).

In some embodiments, the preparation route (S1) is performed in a solvent. In some embodiments, the solvent is selected from one of or a combination of toluene, acetonitrile, dioxane, DMF, DMAc, DMSO, NMP, DMI, ethyl acetate, isopropyl acetate, ethylene dichloride, or the like. In some embodiments, the solvent is toluene.

In some embodiments, the preparation route (S1) is performed under catalysis by a catalyst. In some embodiments, the catalyst is a protonic acid or a Lewis acid.

In some embodiments, the catalyst is selected from hydrochloric acid, HBr, HI, HF, sulfuric acid, phosphoric acid, oxalic acid, methanesulfonic acid, trifluoromethanesulfonic acid, boric acid, benzenesulfonic acid, p-toluenesulfonic acid, or the like. In some embodiments, the catalyst is p-toluenesulfonic acid.

Embodiments of the present disclosure also provide a method for preparing the compound represented by the formula (Ia), the compound is prepared by reacting the compound represented by the formula (II) with a compound represented by a formula (IIIa, and a preparation route (S2) is as follows:

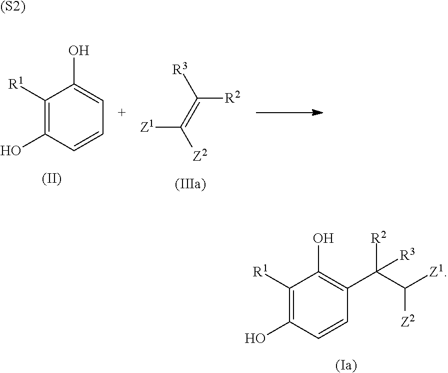

R¹, R², and R³ are defined as defined above, Z¹ and Z² are the same or different, which are independently selected from H, the optionally substituted alkyl group, the optionally substituted cycloalkyl group, and the optionally substituted amide group, and Z¹, Z², and a carbon atom to which Z¹ and Z² are commonly connected, are connected to form a ring.

In some embodiments, a reaction temperature of the preparation route (S2) is within a range of 40-110° C. In some embodiments, the reaction temperature of the preparation route (S2) is within a range of 70-90° C.

In some embodiments, a molar ratio of the compound represented by the formula (II) to the compound represented by the formula (IIIa) is 1:(1-1.5). In some embodiments, the molar ratio of the compound represented by the formula (II) to the compound represented by the formula (IIIa) is 1:(1.01-1.05).

In some embodiments, the preparation route (S2) is performed in a solvent. In some embodiments, the solvent is selected from one of or a combination of toluene, acetonitrile, dioxane, DMF, DMAc, DMSO, NMP, DMI, ethyl acetate, isopropyl acetate, ethylene dichloride, or the like. In some embodiments, the solvent is toluene.

In some embodiments, the preparation route (S2) is performed under catalysis by a catalyst. In some embodiments, the catalyst is a protonic acid or a Lewis acid. In some embodiments, the catalyst is selected from hydrochloric acid, HBr, HI, HF, sulfuric acid, phosphoric acid, oxalic acid, methanesulfonic acid, trifluoromethanesulfonic acid, boric acid, benzenesulfonic acid, p-toluenesulfonic acid, or the like. In some embodiments, the catalyst is p-toluenesulfonic acid.

Embodiments of the present disclosure also provide a method for preparing the compound represented by the formula (V), the compound is prepared by reacting the compound represented by the formula (II) with the compound represented by the formula (IVa), and a preparation route (S3) is as follows:

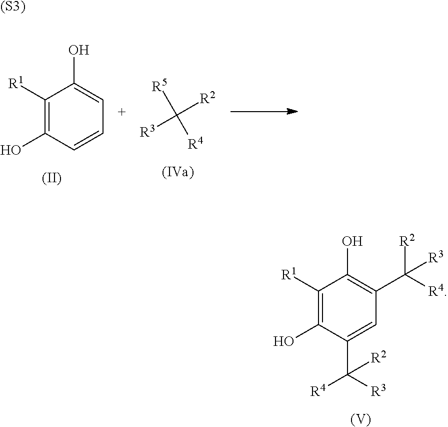

R¹, R², R³, and R⁴ are defined as defined above. R⁵ is selected from a leaving group, which is selected from OH, H₂O, OTs, OMs, Cl, Br, I, or the like.

In some embodiments, a reaction temperature of the preparation route (S3) is within a range of 40-110° C. In some embodiments, the reaction temperature of the preparation route (S3) is within a range of 70-90° C.

In some embodiments, a molar ratio of the compound represented by the formula (II) to the compound represented by the formula (IVa) is 1:(2.0-3.0). In some embodiments, the molar ratio of the compound represented by the formula (II) to the compound represented by the formula (IVa) is 1:(2.01-2.10).

In some embodiments, the preparation route (S3) is performed in a solvent. In some embodiments, the solvent is selected from one of or a combination of toluene, acetonitrile, dioxane, DMF, DMAc, DMSO, NMP, DMI, ethyl acetate, isopropyl acetate, ethylene dichloride, or the like. In some embodiments, the solvent is toluene.

In some embodiments, the preparation route (S3) is performed under catalysis by a catalyst. In some embodiments, the catalyst is a protonic acid or a Lewis acid. In some embodiments, the catalyst is selected from hydrochloric acid, HBr, HI, HF, sulfuric acid, phosphoric acid, oxalic acid, methanesulfonic acid, trifluoromethanesulfonic acid, boric acid, benzenesulfonic acid, p-toluenesulfonic acid, or the like. In some embodiments, the catalyst is p-toluenesulfonic acid.

Embodiments of the present disclosure also provide a method for preparing a compound represented by the formula (Va), the compound is prepared by reacting the compound represented by the formula (II) with the compound represented by the formula (IIIa), and a preparation route (S4) is as follows:

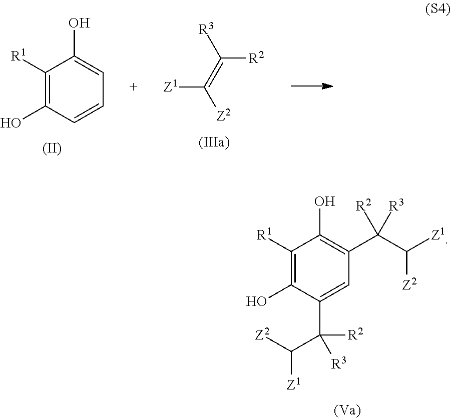

R¹, R², and R³ are defined as defined above. Z¹ and Z² are the same or different, which are independently selected from hydrogen, the optionally substituted alkyl group, the optionally substituted cycloalkyl group, and the optionally substituted amide group, and Z¹, Z², and a carbon atom to which Z¹ and Z² are commonly connected, are connected to form a ring.

In some embodiments, a reaction temperature of the preparation route (S4) is within a range of 40-110° C. In some embodiments, the reaction temperature of the preparation route (S4) is within a range of 70-90° C.

In some embodiments, a molar ratio of the compound represented by the formula (II) to the compound represented by the formula (IIIa) is 1:(2.0-3.0). In some embodiments, the molar ratio of the compound represented by the formula (II) to the compound represented by the formula (IIIa) is 1:(2.01-2.10).

In some embodiments, the preparation route (S4) is performed in a solvent. In some embodiments, the solvent is selected from one of or a combination of toluene, acetonitrile, dioxane, DMF, DMAc, DMSO, NMP, DMI, ethyl acetate, isopropyl acetate, ethylene dichloride, or the like. In some embodiments, the solvent is toluene.

In some embodiments, the preparation route (S4) is performed under catalysis by a catalyst. In some embodiments, the catalyst is a protonic acid or a Lewis acid. In some embodiments, the catalyst is selected from hydrochloric acid, HBr, HI, HF, sulfuric acid, phosphoric acid, oxalic acid, methanesulfonic acid, trifluoromethanesulfonic acid, boric acid, benzenesulfonic acid, p-toluenesulfonic acid, or the like. In some embodiments, the catalyst is p-toluenesulfonic acid.

Embodiments of the present disclosure also provide a method for preparing the compound represented by the formula (VI), the compound is prepared by reacting a compound represented by a formula (I) with a compound represented by a formula (IVb), and a preparation route (S5) is as follows:

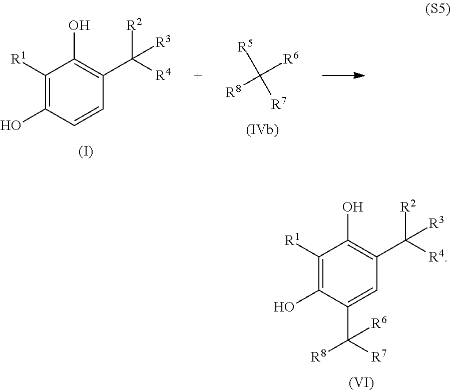

R¹, R², R³, R⁴, R⁶, R⁷, and R⁸ are defined as defined above. R⁵ is selected from a leaving group which is selected from OH, H₂O, OTs, OMs, Cl, Br, I, or the like.

In some embodiments, a reaction temperature of the preparation route (S5) is within a range of 40-110° C. In some embodiments, the reaction temperature of the preparation route (S5) is within a range of 70-90° C.

In some embodiments, a molar ratio of the compound represented by the formula (I) to the compound represented by the formula (IVb) is 1:(1-1.5). In some embodiments, the molar ratio of the compound represented by the formula (I) to the compound represented by the formula (IVb) is 1:(1.01-1.05).

In some embodiments, the preparation route (S5) is performed in a solvent. In some embodiments, the solvent is selected from one or a combination of toluene, acetonitrile, dioxane, DMF, DMAc, DMSO, NMP, DMI, ethyl acetate, isopropyl acetate, ethylene dichloride, or the like. In some embodiments, the solvent is toluene.

In some embodiments, the preparation route (S5) is performed under catalysis by a catalyst, which is a protonic acid or a Lewis acid. In some embodiments, the catalyst is selected from hydrochloric acid, HBr, HI, HF, sulfuric acid, phosphoric acid, oxalic acid, methanesulfonic acid, trifluoromethanesulfonic acid, boric acid, benzenesulfonic acid, p-toluenesulfonic acid, or the like. In some embodiments, the catalyst is p-toluenesulfonic acid.

Embodiments of the present disclosure also provide a method for preparing the compound represented by the formula (VIa), the compound is prepared by reacting the compound represented by the formula (I) with a compound represented by a formula (IIIb), and a preparation route (S6) is as follows:

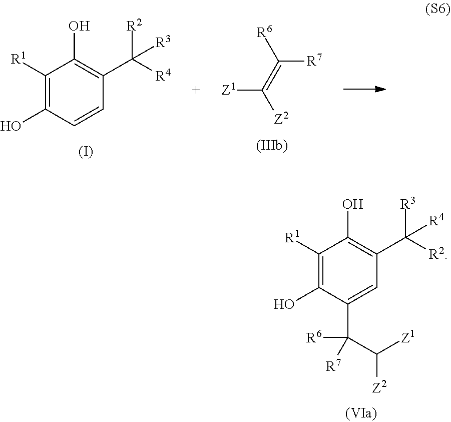

R¹, R², R³, R⁴, R⁶, R⁷, Z¹, and Z² are defined as defined above.

In some embodiments, a reaction temperature of the preparation route (S6) is within a range of 40-110° C. In some embodiments, the reaction temperature of the preparation route (S6) is within a range of 70-90° C.

In some embodiments, a molar ratio of the compound represented by the formula (I) to the compound represented by the formula (IIIb) is 1:(1-1.5). In some embodiments, the molar ratio of the compound represented by the formula (I) to the compound represented by the formula (IIIb) is 1:(1.01-1.05).

In some embodiments, the preparation route (S6) is performed in a solvent. In some embodiments, the solvent is selected from one of or a combination of toluene, acetonitrile, dioxane, DMF, DMAc, DMSO, NMP, DMI, ethyl acetate, isopropyl acetate, ethylene dichloride, or the like. In some embodiments, the solvent is toluene.

In some embodiments, the preparation route (S6) is performed under catalysis by a catalyst. In some embodiments, the catalyst is a protonic acid or a Lewis acid. In some embodiments, the catalyst is selected from hydrochloric acid, HBr, HI, HF, sulfuric acid, phosphoric acid, oxalic acid, methanesulfonic acid, trifluoromethanesulfonic acid, boric acid, benzenesulfonic acid, p-toluenesulfonic acid, or the like. In some embodiments, the catalyst is p-toluenesulfonic acid.

Compounds provided in embodiments of the present disclosure are used in the preparation of pharmaceuticals or cosmetics for treating, preventing, and/or alleviating the hyperpigmentation. In some embodiments, the pharmaceuticals or cosmetics are applied topically to the skin, such as human skin.

Embodiments of the present disclosure also provide a pharmaceutical composition including the compound represented by the formula (A) or a pharmaceutically acceptable salt thereof.

Embodiments of the present disclosure also provide a cosmetic composition including the compound represented by the formula (A) or a pharmaceutically acceptable salt thereof.

In some embodiments, the cosmetic composition is made into a cream, a lotion, a paste, a capsule, a detergent, a foam, a gel, a dispersion, a suspension, a spray, a serum, a mask, or other suitable forms.

In some embodiments, the pharmaceutical composition and/or the cosmetic composition further include one or more additives, such as an antioxidant, an emollient, a humectant, a thickener, a fragrance agent, a preservative, a pigment, a coloring agent, or an opacifier.

The antioxidant is capable of protecting ingredients of the pharmaceutical composition and/or the cosmetic composition from oxidation by an oxidizing agent. The antioxidant includes ascorbyl palmitate, butylated hydroxyanisole, butylated hydroxytoluene, potassium propyl gallate, octyl gallate, dodecyl gallate, phenyl-α-naphthylamine, and tocopherol such as alpha-tocopherol.

The emollient is an agent that softens and smooths the skin. The emollient includes oils, waxes (e.g., microcrystalline wax, polyethylene), triglyceride (e.g., castor oil, cocoa butter, safflower oil, corn oil, olive oil, cod liver oil, almond oil, palm oil, soybean oil), squalene, acetylated monoglyceride, ethoxylated glyceride, fatty acid, alkyl ester of fatty acid, alkenyl ester of fatty acid, fatty alcohols, fatty alcohol ether, ether-ester, lanolin and lanolin derivative, polyol ester, wax ester (e.g. beeswax, vegetable wax), phospholipid, sterols, isopropyl palmitate, glycerol stearate, or the like. In some embodiments, the emollient is almond oil or a fatty alcohol, for example, cetearyl alcohol, stearyl alcohol, and/or myristyl alcohol.

In some embodiments, the emollient is a siloxane. The siloxane includes, but is not limited to, one or any combination of a dimethicone, a methyl cyclosiloxane, a phenyl polytrimethyl siloxane, a phenyl polydimethyl siloxane, a cetyl dimethicone, a stearyl dimethicone, an amino-capped dimethicone, a C30-45 alkyl dimethicone, a C30-45 alkyl methyl siloxane, a cetearyl stearyl methyl siloxane, a dimethicone copolyol, a cyclopentylsiloxane, a cyclohexylsiloxane, etc. In some embodiments, the emollient is an amino-capped dimethicone.

The humectant is capable of increasing and maintaining moisture in the skin. The humectant includes propylene glycol, butylene glycol, polyethylene glycol (PEG) (e.g., PEG-4 to PEG-32), glycerol, sorbitol, xylitol, maltitol, mannitol, polydextrose, hyaluronic acid and salts thereof (e.g., sodium or potassium salts), urea, aloe vera juice, honey, and the like.

The thickener is capable of increasing a viscosity and consistency of the composition. The thickener includes a lipid thickener such as cetyl alcohol, stearyl alcohol, myristyl alcohol, Brazilian carnauba wax, or stearic acid; a naturally derived thickener such as cellulose derivatives, hydroxyethyl cellulose, guar gum, locust bean gum, xanthan gum, or gelatin; a mineral thickener such as silicon dioxide, bentonite, or aluminum magnesium silicate; a synthetic thickener such as carbomer; and an ionic thickener, such as NaCl.

The fragrance agent is capable of increasing the fragrance of the composition. The fragrance agent includes peppermint, rose oil, rose water, aloe vera juice, clove oil, menthol, camphor, eucalyptus oil, and other plant extracts. A masking agent is used in order to eliminate certain odors from the composition.

The preservative is capable of protecting the composition from degradation. The preservative includes one or a mixture of phenoxyethanol, methyl para-hydroxybenzoate, benzalkonium chloride, benzethonium chloride, propyl para-hydroxybenzoate, benzoic acid, benzyl alcohol, and the like. In some embodiments, the preservative is phenoxyethanol, methyl para-hydroxybenzoate, or a mixture thereof.

The pigment or coloring agent is capable of changing the color of the composition, and the opacifier is capable of making a clarified or transparent composition opaque. In some embodiments, the pigment or coloring agent or opacifier includes an insoluble photoprotective dye, such as a finely dispersed metal oxide or a metal salt, etc. Titanium dioxide is selected to obtain a white composition. In some embodiments, the metal oxide also includes a zinc oxide, an iron oxide, a zirconium oxide, a silicon oxide, a manganese oxide, an aluminum oxide, and a cerium oxide and a mixture thereof. The metal salt includes a silicate (talc), a barium sulfate, or a zinc stearate, etc. In some embodiments, particles of the metal oxide and the metal salt have an average diameter of less than 100 nm. In some embodiments, particles of the metal oxide and the metal salt have an average diameter of 5-50 nm. In some embodiments, particles of the metal oxide and the metal salt have an average diameter of 15-30 nm. In some embodiments, particles of the metal oxide and the metal salt are spherical, elliptical, or other shapes. In some embodiments, the pigment is treated on the surface, such as hydrophilized or hydrophobic. For example, a coated titanium dioxide. In some embodiments, a hydrophobic coating agent for the surface treatment includes an organosilicon, such as a trialkoxyoctylsilane or a trisiloxane.

In some embodiments, the photoprotective dye is selected from a microfine titanium dioxide, a zinc oxide, and a microfine zinc oxide. When the titanium dioxide is selected as the photoprotective dye, the total amount of the titanium dioxide is 0.1%-10.0% by weight of the composition. When the zinc oxide is selected as the photoprotective dye, the total amount of the zinc oxide is 0.1%-10.0% by weight of the composition. When one or more triazine organic pigments are selected, the total amount of the triazine organic pigments is 0.1%-10.0% by weight of the composition.

In some embodiments, the pharmaceutical composition or the cosmetic composition further includes at least one brightening agent, e.g., sclareolide.

The compound represented by the formula (A) provided by embodiments of the present disclosure is also used in a decolorant, a whitening agent, a bleaching agent, or a brightening agent, or a cosmetic for the skin.

The cosmetic composition provided by embodiments of the present disclosure is also used in a composition of the decolorant, the whitening agent, the bleaching agent, the brightening agent, and the cosmetic for topical application to the skin.

The compound represented by the formula (A) shown in the embodiments of the present disclosure is used to prepare a cosmetic composition which is used to decolorize, whiten, bleach, or brighten the skin, such as human skin.

Embodiments of the present disclosure also provide a method for administering an effective amount of the compound or the cosmetic composition to the skin of a person in need thereof, the method being capable of decoloring, whitening, bleaching, or brightening the skin, such as human skin.

Embodiments of the present disclosure also provide a pharmaceutical composition that is used in the treatment of hyperpigmentation disorders.

Embodiments of the present disclosure also provide a dermatological composition that is used for treating hyperpigmentation disorders, the dermatological composition being applied topically to the skin, such as human skin.

Embodiments of the present disclosure also provide a method for administering an effective amount of the compound or the pharmaceutical composition to the skin of a person in need thereof, the method being capable of treating a hyperpigmentation disorder of the skin.

The hyperpigmentation disorder manifests as hyperpigmentation and includes small moles, acanthosis nigricans, freckles, post-inflammatory hyperpigmentation, and hyperpigmentation induced by drugs, chemicals, or sunlight.

Embodiments of the present disclosure provide a resorcinol compound, which is used as an antioxidant to inhibit or reduce oxidative stress, such as an oxidative stress induced by UV. In some embodiments, the resorcinol compound is used as the antioxidant to inhibit an oxidative stress in the skin.

Embodiments of the present disclosure also provide a method for inhibiting or reducing the oxidative stress, particularly the oxidative stress induced by UV, the method including administering or topically administering an effective amount of the resorcinol compound to a person in need thereof.

When applied to the human skin, the combination of the resorcinol compound and UV-filtering substance is well tolerated, which does not cause reddening, whitening, or browning of the skin, is non-irritating, does not dry out the skin or form a moist, scaly, powdery, or sticky film, and does not chafe the skin. The UV filtering substance is a UV-A filtering substance, a UV-B filtering substance, a photoprotective dye, or a mixture thereof. The UV filtering substance is an organic substance (e.g., a light filtering substance) that is, for example, liquid or crystalline at a room temperature, capable of absorbing UV radiation and releasing the absorbed energy in a form of long-wave radiation, such as heat. In some embodiments, the content of the UV filtering substance is within a range of 0.05%-50% by weight of the composition. In some embodiments, the content of the UV filtering substance is within a range of 0.5%-40% by weight of the composition.

The UV filtering substance is oil soluble or water soluble. The oil-soluble UV filtering substance includes: 3-benzylidene camphor and its derivatives, such as 3-(4-methylbenzylidene) camphor; 4-aminobenzoic acid derivatives, such as 2-ethylhexyl 4-(dimethylamino) benzoate, 2-octyl 4-(dimethylamino) benzoate, and amyl 4-(dimethylamino) benzoate; esters of cinnamic acid, such as 2-ethylhexyl 4-methoxycinnamic acid, 4-methoxy propyl cinnamate, isoamyl 4-methoxycinnamate, 2-ethylhexyl 2-cyano-3,3-diphenylpropenoate (octocrylene); esters of salicylic acid, such as 2-ethylhexyl salicylate, humulanic acid ester, and menthyl salicylate; and benzophenone derivatives, such as 2,2′-dihydroxy-4,4′-dimethoxybenzophenone, 2-hydroxy-4-methoxy-benzophenone, 2,2′-dihydroxy-4-methoxybenzophenone; benzyl malonates, such as bis-2-ethylhexyl 4-methoxybenzyl malonate; triazine derivatives, such as, 2,4,6-tris(p-2-ethylhexylanilino)-1,3,5-triazine and bis(2-octylbutylamino)triazinone; benzoylmethane derivatives, such as 4-tert-butyl-4′-methoxydibenzoylmethane; carbonyl-containing polycyclic compounds, such as ketotricyclic (5.2.1.0) decane derivatives.

Examples of the suitable water-soluble material include: 2-phenylbenzimidazole-5-sulfonic acid and its alkali metal salts, alkaline-earth metal salts, ammonium salts, alkylammonium salts, alkanol salts, and gluconium salts; disodium salts of 2,2′-bis-(1,4-phenylene) 1H-benzimidazole-4,6-disulfonic acid; sulfonic acid derivatives of benzophenone, such as 2-hydroxy-4-methoxybenzophenone-5-sulfonic acid and salts thereof; sulfonic acid derivatives of 3-benzylidene camphor, such as benzylidene camphor sulfonic acid and 2-methyl-5-(2-oxo-3-bornylidene)sulfonic acid and salts thereof.

The UV-A filtration substance includes of benzoylmethane derivative, such as 4-tert-butyl-4′-methoxy-dibenzoylmethane, hexyl 2-(4-diethylamino-2-hydroxybenzoyl)-benzoate, 1-phenyl-3-(4′-isopropylphenyl)propane-1,3-dione, and alkenylamine compound. In some embodiments, the UV-A and UV-B filtration substances may be mixed for use, e.g., by a combination of a diphenylmethane derivative and/or a benzoylmethane derivative, 4-tert-butyl-4′-methoxy-dibenzoylmethane, and a cinnamic acid derivative, which is selected from 2-ethylhexyl 2-cyano-3,3-diphenylpropenoate (octocrylene), 4-methoxycinnamic acid 2-ethylhexyl, isoamyl 4-methoxycinnamate and/or propyl 4-methoxycinnamate. The cinnamate derivative of the combination may be replaced by a water-soluble filter substance, such as 2-phenylbenzimidazole-5-sulfonic acid and its alkali metal salt, alkaline-earth metal salt, ammonium salt, alkyl ammonium salt, alkanol salt and gluconium salt, 4-(2-oxo-3-borneolylmethyl)benzenesulfonic acid or 2-methyl-5-(2-oxo-3-borneolyl)sulfonic acid and its alkali metal salt, alkaline-earth metal salt, ammonium salt, alkylammonium salt, alkanol salt and gluconium salt.

The pharmaceutical composition and/or the cosmetic composition of the present disclosure contain at least one additional UV-absorbing substance, which is selected from camphor benzalkonium methosulfate, p-phenylenedimethylene-dibenzyl sulfonic acid and salts thereof, homanthenol salicylate, benzylidene camphorsulfonic acid and salts thereof, 2-cyano-3,3-diphenylpropenoic acid-2-ethylhexyl ester, ethyl para-aminobenzoate, isoamyl para-methoxycinnamate, 2-phenylbenzimidazole sulfonic acid and salts thereof, 2,4,6-tris (p-2-ethylhexylaniline)-1,3,5-triazine, 2-(2H-benzotriazol-2-yl)-4-methyl-6-(2-methyl-3-(1,3, 3,3-tetramethyl-1-(trimethylsilyl-oxo)-disiloxanyl)-propyl)phenol, methylenebis-benzotriazolyltetramethylbutyl phenol, 4,4′-[(6-[4-(1,1-dimethyl)-aminocarbonyl]phenylamino)-1,3,5-triazine-2,4-diyl]bisimino]-di-(2-ethylhexyl benzoate), 3-(4′-methylbenzylidene)-D, L-camphor, 3-benzylidene camphor, 2-ethylhexyl salicylic acid, 4-dimethylaminobenzoic acid-2-ethylhexyl ester, 4-hydroxy-4-methoxybenzophenone-5-sulfonic acid and salts thereof, benzylidene malonate-polysiloxane, menthyl o-aminobenzoate, polyacrylamidomethylbenzylidene camphor, 2-ethylhexyl 4-methoxycinnamate, or a mixture thereof.

When applied to human skin, the composition of the present disclosure is used with a brightening agent to enhance a mitigating effect on the hyperpigmentation. The brightening agent includes: kojic acid derivatives (e.g., kojic acid dipalmitate), arbutin, ascorbic acid and ascorbic acid derivatives (e.g., magnesium ascorbylphosphate), hydroquinone and hydroquinone derivatives, sclareolide, amino acids (e.g., cyclohexylcarbamates, N-acetyltyrosine and its derivatives, undecylenoyl phenylalanine), sulfur-containing molecules (e.g., glutathione, cysteine, thiourea derivatives, lipoic acid), alpha-hydroxy acids (e.g., citric acid, lactic acid, malic acid and salts and esters thereof), gluconic acid, chromone derivatives (e.g., aloe vera bitters), 1-aminoethyl hypophosphite, flavonoids, ellagic acid, nicotinamide, clavulin and its derivatives, triterpenes (e.g., hawthornic acid), stanols (e.g., ergosterol), benzofurans (e.g., ligustilide), 4-vinylguaiacol, 4-ethylguaiacol, zinc salts (e.g. zinc chloride or zinc gluconate), diacids (e.g. octadecenedioic acid and/or azelaic acid), inhibitors of nitrogen oxide synthesis (e.g. L-nitroarginine and its derivatives, 2,7-dinitroindazole, or thio-L-citrulline), metal chelators (e.g. α-hydroxy fatty acids, phytates, cholic acids, bile extracts, humic acids, EGTA, EDTA and its derivatives), soy milk and its extracts, retinoic acid, serine protease inhibitors, other synthetic or extracted natural active ingredients (e.g., plant extracts, such as bearberry extracts, grape extracts, mulberry fruit extracts, jicamam extracts, rice extracts, papaya extracts, turmeric extracts, balsam extracts, licorice root extracts (e.g., glycyrrhizin or glycyrrhizin chalcone A), cinnamon extract, extracts of the genus Rumex, extracts of the genus Pinus (e.g., pine), extracts of the genus Grapevine or stilbene derivatives isolated or concentrated therefrom, extracts of Saxifrage, extracts of Scutellaria baicalensis, microalgae extracts (e.g., extracts of the genus Tetraselmis tetrathele), and extracts of Ginseng.

The present disclosure is described in detail below by means of examples. It may be understood, however, that the relevant examples are not intended to limit the scope of protection of the present disclosure in any way.

### EXAMPLES

The amounts of reactants and products were measured by liquid chromatography (Agilent HPLC 1260).

The conversion rate and selectivity of a reaction were calculated by the following equations (I) and (2), respectively:

$$( 1 )$$

$$Conversion rate = \frac{\begin{matrix}molar amount of raw material input - molar amount of raw \\ material remaining in the product\end{matrix}}{molar amount of raw material input} \times 100 \%$$

$$Selectivity = \frac{actual molar amount of a target product}{theoretical molar amount of the target product} \times 100 \% . \qquad ( 2 )$$

Unless otherwise specified, the raw material used is commercially available and the room temperature is 25±5° C.

### Example 1

### Preparation of a Compound Represented by a Formula (I-a)

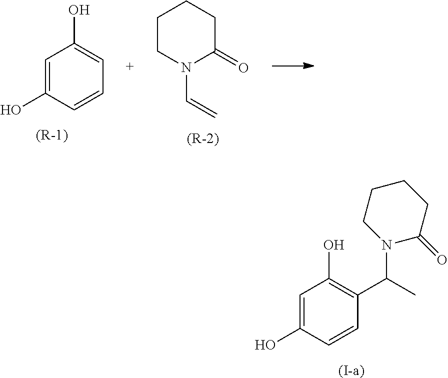

250 g of toluene, 1.0 g of p-toluenesulfonic acid as a catalyst, 110 g of resorcinol (R-1), and 125 g of N-vinylpiperidone (R-2) were added into a 1000 mL four-necked flask and heated, and the reaction was carried out at 90° C. for 10 h. When the reaction was detected to be complete using the liquid chromatography, the solvent was stripped off under reduced pressure, and a mixed solvent of ethyl acetate and petroleum ether was added for recrystallization to obtain the compound represented by the formula (I-a), with a yield of 90% and a purity of 99%.

### Example 2

### Preparation of a Compound Represented by a Formula (I-b)

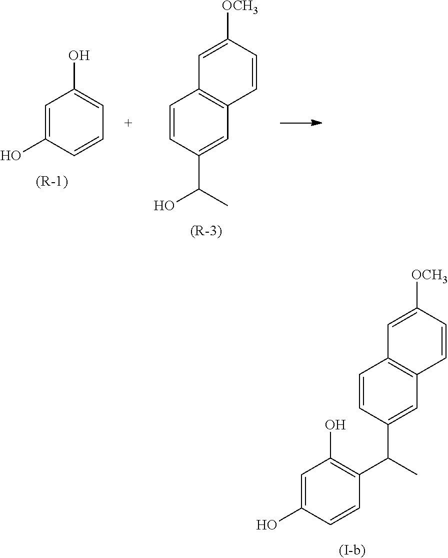

250 g of toluene, 1.0 g of p-toluenesulfonic acid as a catalyst, 110 g of resorcinol (R-1), and 202 g of 6-methoxy-2-naphthalene ethanol (R-3) were added into a 1000 mL four-necked flask and heated, and the reaction was carried out at 90° C. for 10 h. When the reaction was detected to be complete using the liquid chromatography, the solvent was stripped off under reduced pressure, and a mixed solvent of ethyl acetate and petroleum ether was added for recrystallization to obtain the compound represented by the formula (I-b), with a yield of 92% and a purity of 99%.

### Example 3

### Preparation of a Compound Represented by a Formula (V-c)

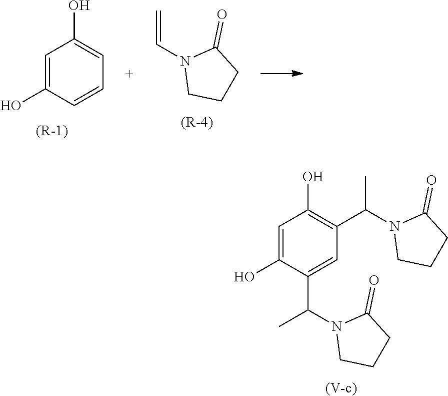

250 g of toluene, 1.0 g of p-toluenesulfonic acid as a catalyst, 110 g of resorcinol (R-1), and 222 g of N-vinylpyrrolidone (R-4) were added into a 1000 mL four-necked flask and heated, and the reaction was carried out at 90° C. for 10 h. When the reaction was detected to be complete using the liquid chromatography, the solvent was stripped off under reduced pressure, and a mixed solvent of ethyl acetate and petroleum ether was added for recrystallization to obtain the compound represented by the formula (V-c), with a yield of 85% and a purity of 96%.

### Example 4

### Preparation of a Compound Represented by a Formula (V-a)

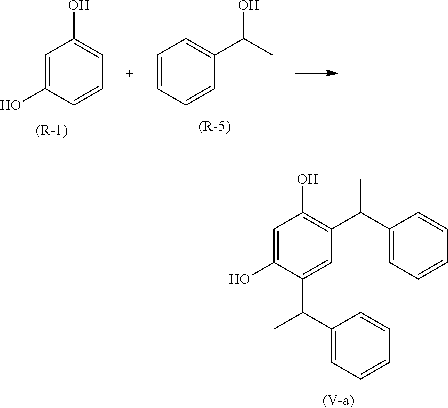

250 g of toluene, 1.0 g of p-toluenesulfonic acid as a catalyst, 110 g of resorcinol (R-1), and 244 g of 1-phenylethanol (R-5) were added into a 1000 mL four-necked flask and heated, and the reaction was carried out at 90° C. for 10 h. When the reaction was detected to be complete using the liquid chromatography, the solvent was stripped off under reduced pressure, and the compound was separated and purified by a silica gel column (using a mixed solvent of ethyl acetate and petroleum ether for elution), and the compound represented by the formula (V-a) was obtained by concentrating and removing the solvent, with a yield of 82% yield and a purity of 97%.

### Example 5

### Preparation of a Compound Represented by a Formula (V-c)

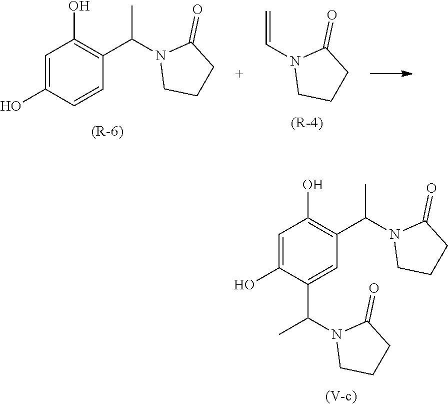

150 g of toluene, 0.50 g of p-toluenesulfonic acid as a catalyst, 110 g of resorcinol pyrrolidone derivative (R-6), and 56 g of N-vinylpyrrolidone (R-4) were added into a 1000 mL four-necked flask and heated, and the reaction was carried out at 90° C. for 10 h. When the reaction was detected to be complete using the liquid chromatography, the solvent was stripped off under reduced pressure, and a mixed solvent of ethyl acetate and petroleum ether was added for recrystallization to obtain the compound represented by the formula (V-c), with a yield of 84% and a purity of 97%.

### Example 6

### Preparation of a Compound Represented by a Formula (V-a)

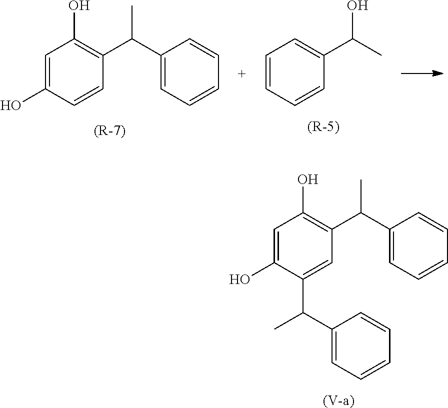

250 g of toluene, 1.0 g of p-toluenesulfonic acid as a catalyst, 214 g of resorcinol benzene-substituted ethyl derivative (R-7), and 122 g of 1-phenylethanol (R-5) were added into a 1000 mL four-necked flask and heated, and the reaction was carried out at 90° C. for 10 h. When the reaction was detected to be complete using the liquid chromatography, the solvent was stripped off under reduced pressure, the compound was separated and purified by a silica gel column (using a mixed solvent of ethyl acetate and petroleum for elution), and the compound represented by the formula (V-a) was obtained by concentrating and removing the solvent, with a yield of 80% and a purity of 95%.

Compounds in the following Tables 1-3 were obtained by preparation with reference to methods of the above embodiments. Table 1 shows the compounds with a structural formula (I):

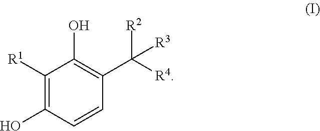

TABLE 1
Compounds with the structural formula (I)

| Serial | | | | | |
|---|---|---|---|---|---|
| number | R¹ | R² | R³ | R⁴ | m/z |
| I-a | H | 2-oxopiperidin-1-yl | H | CH₃ | 235.1 |
| I-b | H | 6-methoxy-2-naphthalenyl | H | CH₃ | 294.1 |
| I-c | H | 2-oxopiperidin-1-yl | H | CH₂CH₃ | 249.1 |
| I-d | H | 2-oxopyrrolidin-1-yl | H | CH₂CH₃ | 235.1 |
| I-e | H | 2-oxopiperidin-1-yl | H | CH₂CH₂CH₃ | 263.2 |
| I-f | H | 2-oxopyrrolidin-1-yl | H | CH₂CH₂CH₃ | 249.1 |
| I-g | H | 2-oxoazepan-1-yl | H | CH₃ | 249.1 |
| I-h | H | 2-oxopyrrolidin-1-yl | H | CH₃ | 221.1 |
| I-i | H | 2-oxoazepan-1-yl | H | CH₂CH₃ | 263.2 |
| I-j | H | 2-oxoazepan-1-yl | H | CH₂CH₂CH₃ | 277.2 |
| I-k | H | 2-oxopyrrolidin-1-yl | H | H | 207.1 |
| I-l | H | 2-oxopiperidin-1-yl | H | H | 221.1 |
| 1-m | H | 2-oxoazepan-1-yl | H | H | 235.1 |
| I-n | H | acetamido | H | CH₃ | 195.1 |
| I-o | H | 2-carboxy-5-oxopyrrolidin-1-yl | H | CH₃ | 265.1 |
| I-p | H | 2-(methoxycarbonyl)-5-oxopyrrolidin- | H | CH₃ | 279.1 |
| | | 1-yl | | | |
| I-q | H | 2-(methylcarbamoyl)-5-oxopyrrolidin- | H | CH₃ | 278.1 |
| | | 1-yl | | | |
| I-r | H | formamido | H | CH₃ | 181.1 |
| I-s | CH₃ | 2-oxopiperidin-1-yl | H | CH₃ | 249.1 |
| I-t | CH₃ | 2-oxoazepan-1-yl | H | CH₃ | 263.2 |
| I-u | CH₃ | 2-oxopyrrolidin-1-yl | H | CH₃ | 235.1 |
| | | | H | | |
| I-v | H | phenyl | H | 1-n-dodecyl-2- | 479.3 |
| | | | H | oxoazepan-3-yl | |
| I-w | H | phthalimido | H | CH₃ | 283.1 |

Table 2 shows compounds with a structural formula (VI):

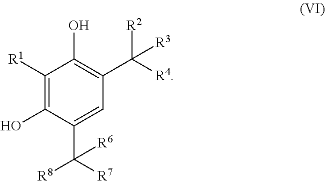

TABLE 2
Compounds with the structural formula (VI)

| Serial | | | | | | | | |
|---|---|---|---|---|---|---|---|---|
| number | R¹ | R² | R³ | R⁴ | R⁶ | R⁷ | R⁸ | m/z |
| V-a | H | phenyl | H | CH₃ | phenyl | H | CH₃ | 318.2 |
| V-b | H | 2-oxoazepan-1-yl | H | CH₃ | 2-oxoazepan-1-yl | H | CH₃ | 388.2 |
| V-c | H | 2-oxopyrrolidin-1-yl | H | CH₃ | 2-oxopyrrolidin-1-yl | H | CH₃ | 332.2 |
| V-d | H | 2-oxopiperidin-1-yl | H | CH₃ | 2-oxopiperidin-1-yl | H | CH₃ | 360.2 |
| V-e | CH₃ | 2-oxopiperidin-1-yl | H | CH₃ | 2-oxopiperidin-1-yl | H | CH₃ | 374.2 |
| V-f | H | acetamido | H | CH₃ | acetamido | H | CH₃ | 280.1 |
| V-g | H | formamido | H | CH₃ | formamido | H | CH₃ | 252.1 |
| V-h | H | 2-carboxy-5- | H | CH₃ | 2-carboxy-5- | H | CH₃ | 420.2 |
| | | oxopyrrolidin-1-yl | | | oxopyrrolidin-1-yl | | | |
| VI-a | H | 2-oxopyrrolidin-1-yl | H | CH₃ | phenyl | H | CH₃ | 325.2 |

TABLE 3
NMR data of compounds (I-a) to (I-g) and compounds (v-a) and (v-c)

| Serial | | |
|---|---|---|
| number | Structure of Compound | H-NMR |
| I-a | 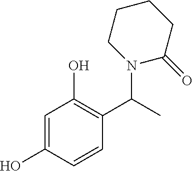 [SMILES: CC(c1ccc(O)cc1O)N1CCCCC1=O] | ¹H NMR (500 MHz, DMSO-d6) δ 1.33 (d, J = 7.1 Hz, 3H), 1.49-1.62 (m, 4H), 2.16-2.27 (m, 2H), 2.70- 2.75(m,1H),3.04-3.09 (m,1H), 5.71(q, J = 7.1 Hz, 1H), 6.18-6.21 (m,2H), 7.01 (d, J = 8.1 Hz, 1H), 9.29 (s, 1H), 9.43 (s, 1H). |
| I-b | 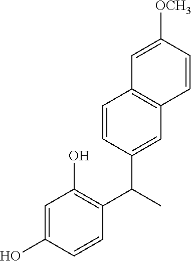 [SMILES: COc1ccc2cc(C(C)c3ccc(O)cc3O)ccc2c1] | ¹H NMR (500 MHz, DMSO-d6) δ 1.53 (d, J = 7.3 Hz, 3H), 3.84 (s, 3H), 4.45 (q, J = 7.3 Hz, 1H), 6.16 (dd, J = 8.5, 2.5 Hz, 1H), 6.27 (d, J = 2.5 Hz, 1H), 6.85 (d, J = 8.5 Hz, 1H), 7.10 (dd, J = 9.0, 2.5 Hz, 1H), 7.23 (d, J = 2.0 Hz, 1H), 7.30 (dd, J = 8.5, 2.0 Hz, 1H), 7.61 (s, 1H), 7.67 (d, J = 8.5 Hz, 1H), 7.74 (d, J = 9.0 Hz, 1H), 9.01 (s, 1H), 9.18 (s, 1H). |
| I-c | 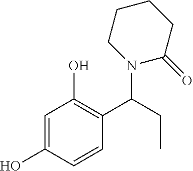 [SMILES: CCC(c1ccc(O)cc1O)N1CCCCC1=O] | ¹H NMR (500 MHz, DMSO-d6) δ 0.81 (t, J = 7.0 Hz, 3H), 1.46-1.72 (m, 4H), 1.76-1.84 (m, 1H), 1.86-1.95 (m, 1H), 2.20-2.34 (m, 2H), 2.82-2.86(m, 1H), 3.08-3.13 (m, 1H), 5.47 (t, J = 7.5 Hz, 1H), 6.19-6.21 (m, 2H), 7.01 (d, J = 8.0 Hz, 1H), 9.23 (s, 1H), 9.36 (s, 1H). |
| I-d | 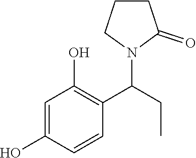 [SMILES: CCC(c1ccc(O)cc1O)N1CCCC1=O] | ¹H NMR (500 MHz, DMSO-d6) δ 0.79 (t, J = 7.5 Hz, 3H), 1.76-1.91 (m, 4H), 2.14-2.28 (m, 2H), 2.95-2.99 (m, 1H), 3.26-3.30 (m, 1H), 5.04 (t, J = 7.5 Hz, 1H), 6.19(dd, J = 8.5, 2.5 Hz, 1H), 6.25 (d, J = 2.5 Hz, 1H), 6.97(d, J = 8.5 Hz, 1H), 9.19 (s, 1H), 9.21 (s, 1H). |
| I-e | 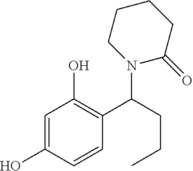 [SMILES: CCCC(c1ccc(O)cc1O)N1CCCCC1=O] | ¹H NMR (500 MHz, DMSO-d6) δ 0.89 (t, J = 7.0 Hz, 3H), 1.15-1.25 (m, 2H), 1.45-1.52 (m, 1H), 1.58-1.77 (m, 4H), 1.85-1.93 (m, 1H), 2.19-2.34(m, 2H), 2.83-2.87 (m, 1H), 3.08-3.14 (m, 1H), 5.59 (t, J = 7.5 Hz, 1H), 6.19-6.21 (m, 2H), 7.02 (d, J = 9.0 Hz, 1H), 9.23 (s, 1H), 9.36 (s, 1H). |
| I-f | 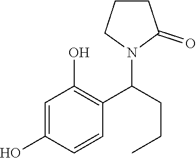 [SMILES: CCCC(c1ccc(O)cc1O)N1CCCC1=O] | ¹H NMR (500 MHz, DMSO-d6) δ 0.88 (t, J = 7.5 Hz, 3H), 1.18-1.24 (m, 2H), 1.69-1.91 (m, 4H), 2.13-2.26 (m, 2H), 2.95-3.00 (m, 1H), 3.26-3.30 (m, 1H), 5.16 (t, J = 7.5 Hz, 1H), 6.19(dd, J = 8.0, 2.5 Hz, 1H), 6.25 (d, J = 2.5 Hz, 1H), 6.97(d, J = 8.0 Hz, 1H), 9.19 (s, 1H), 9.20 (s, 1H). |
| I-g | 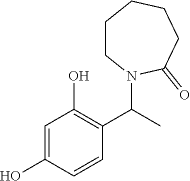 [SMILES: CC(c1ccc(O)cc1O)N1CCCCCC1=O] | ¹H NMR (500 MHz, DMSO-d6) δ 1.11-1.21 (m, 1H), 1.21-1.28 (m, 1H), 1.30 (d, J = 7.0 Hz, 3H), 1.46-1.61 (m, 4H), 2.37-2.46 (m, 2H), 3.02-3.18 (m, 2H), 5.64 (q, J = 7.0 Hz, 1H), 6.19-6.22 (m, 2H), 7.02 (d, J = 8.0 Hz, 1H), 9.19 (s, 1H), 9.21 (s, 1H). |
| V-a | 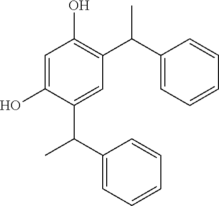 [SMILES: CC(c1ccccc1)c1cc(C(C)c2ccccc2)c(O)cc1O] | ¹H NMR (500 MHz, DMSO-d6) δ1.40-1.44 (m, 6H), 4.31 (q, J = 7.0 Hz, 2H), 6.29 (s, 1H), 6.90 (d, J = 6.0 Hz, 1H), 7.08-7.22 (m, 10H), 8.96 (s, 2H). |
| V-c | 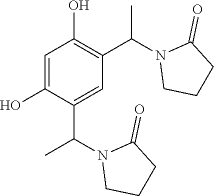 [SMILES: CC(c1cc(C(C)N2CCCC2=O)c(O)cc1O)N1CCCC1=O] | ¹H NMR (500 MHz, DMSO-d6) δ1.36 (d, J = 7.5 Hz, 6H), 1.78-1.89 (m, 4H), 2.15-2.25 (m, 4H), 2.85-2.94 (m, 2H), 3.26-3.35 (m, 2H), 5.25 (m, 2H), 6.31 (s, 1H), 6.98 (s, 1H), 9.30 (s, 1H), 9.30(s, 1H). |

### Example 7: Evaluation of Tyrosinase Inhibitory Activity, Cytotoxicity and Melanin Synthesis Inhibition

### Test Method A: Test for Inhibitory Activity of Samples Against Tyrosinase by Absorbance Method

The compounds of the embodiments of the present disclosure were formulated into solutions with five concentrations of 5 μg/mL, 3 μg/mL, 1 μg/mL, 0.5 μg/mL, 0.01 μg/mL, respectively. 50 μL of each of the above solutions with five concentrations were added into 950 μL of phosphate buffer solution with pH of 6.8 to obtain 1 mL of a solution, then 1 mL of tyrosine with a concentration of 0.1 mg/mL was added into the solution, and 1 mL of tyrosinase solution (200 U/mL) was added into the solution to obtain a mixed solution, the tyrosinase solution was prepared with phosphate buffer solution with pH of 6.8 and tyrosinase. The mixed solution was incubated at 37° C. for 20 min, and the absorbance value was measured at 490 nm. Positive control kojic acid was formulated into solutions with concentrations of 25 μg/mL, 20 μg/mL, 10 μg/mL, 7.5 μg/mL, 5 μg/mL, and 2.5 μg/mL, respectively.

The inhibitory activity of the compounds on tyrosinase was calculated by the following equation (3):

$$I = [ ( A 2 - A 1 ) - ( B 2 - B 1 ) ] / ( A 2 - A 1 ) * 100 \% \qquad ( 3 )$$

Where I denotes an inhibition rate of tyrosinase, A1 denotes an absorbance value of the tyrosinase solution at 0 min in the case of the compound and positive control kojic acid not being added, A2 denotes an absorbance value of the tyrosinase solution at 20 min in the case of the compound and positive control kojic acid not being added, B1 denotes an absorbance value of the tyrosinase solution at 0 min in the case of the compound and positive control kojic acid being added, and B2 denotes an absorbance value of the tyrosinase solution at 20 min in the case of the compound and positive control kojic acid being added.

The test results show that the compounds of the embodiments of the present disclosure have a good inhibitory effect on tyrosinase when the Half Maximal Inhibitory Concentration (IC50) of the compounds is 10 μM, compounds (I-a), (I-b), (I-c), (I-d), (I-e), (I-f), (I-g), (I-h), (V-a), (V-b), and (V-c) have a good inhibitory effect at the concentration of 0.05 μM-1 μM, and the IC50 of the positive control sample kojic acid is 47.0 μM. The above results indicate that the compounds of the embodiments of the present disclosure all have a strong inhibitory activity on tyrosinase. The compounds in the embodiments of the present disclosure can be used alone or in combination as tyrosinase inhibitors for inhibiting enzymatic browning of fruits and vegetables, for preparing whitening cosmetics, and for preventing and treating human pigmentation disorders caused by excessive melanin, melanoma and other diseases that require suppression of tyrosinase activity.

### Test Method B: B-1 Cytotoxicity Test

a) Cell inoculation: human melanoma cells (G361) were inoculated into 96-well plates at a density of 1×10⁴ cells/well, and the 96-well plates were placed in an incubator (37° C., 5% CO₂) overnight for incubation.
b) Experimental grouping: the experiments were set up with a zeroing group, a control group, a positive control group, and a sample group. In the sample group, each sample was set up with different concentration gradients, and 3 replicate wells were set up under each concentration gradient.
c) Solution preparation: different concentrations of sample working solution were prepared according to the concentrations in Table 4.
d) Drug administration: a drug administration was performed until the cell plating rate in the 96-well plate reaches 40%-60%. In the control group, 200 μL of culture medium containing 10% PBS (Gibco) and phenylethyl resorcinol was added to each well; in the positive control group, 200 μL of culture medium containing 10% DMSO was added to each well; in the sample group, 200 μL of culture medium containing the corresponding concentration of sample was added to each well; in the zeroing group, no cell was inoculated and only 200 μL of cell culture medium was added. After the drug administration completed, the 96-well plates were placed in an incubator (37° C., 5% CO₂) for incubation.
e) Detection: after incubation for 24 h, the supernatant was discarded, and MTT working solution (0.5 mg/mL) was added and incubated at 37° C. in the dark for 4 h. After incubation, the supernatant was discarded, and 100 μL DMSO was added into each well and the OD value was obtained at 490 nm.
f) Relative cell viability was calculated by the following equation (4):

$$Relative cell viability = \frac{OD of a sample well - OD of a zeroing well}{OD of a positive control well - OD of the zeroing well} * 100 \% \qquad ( 4 )$$

TABLE 4
Test data of relative cell viability

| | Sample concentration (g/mL) | | | | | |
|---|---|---|---|---|---|---|
| | 0.5 | 0.25 | 0.125 | 0.03125 | 0.015625 | 0.007813 |
| Relative cell | 107.9874 | 120.9409 | 113.8122 | 107.8966 | 120.6987 | 117.8165 |
| viability of I-g (%) | | | | | | |
| Relative cell | 96.77 | 106.07 | 100.10 | 101.15 | 114.68 | 106.32 |
| viability of the | | | | | | |
| control group (%) | | | | | | |

The relative cell viability data indicates that the compounds of the embodiments of the present disclosure have low cytotoxicity.

### B-2 Inhibition Test of Cell Melanin Synthesis

According to cytotoxicity test data, an inhibition test of cell melanin synthesis was performed within the range allowed by cytotoxicity. The range allowed by cytotoxicity is a range of sample concentration corresponding to a relative cell viability more than 60% in the cytotoxicity test. The samples were grouped according to different concentrations, and a test index was melanin content, a test model was melanin cell, and a detection method is a chromogenic method. Cells at logarithmic growth phase were collected, and the cells were inoculated into 24-well plates at a cell density of 2×10⁵ cells/well. After 24 h of incubation in the incubator (37° C., 5% CO₂), according to the results of cytotoxicity, the drug was added according to the concentration requirements. The untreated cells were used as a blank control, and three parallel controls were set up in each group. After the drug added, the incubation was continued in the incubator (37° C., 5% CO₂) for 48 h. The supernatant was discarded, 1 mL of 1 M NaOH containing 10% DMSO was added, and the plate was placed in a thermostatic oven for incubation at 60° C. for 1 h. After restoring to a room temperature, 200 μL mixed solution of each well was transferred to a 96-well plate, and 1 M NaOH containing 10% DMSO was used as a blank control. The absorbance value was read at 405 nm, and the inhibition rate of cellular melanin synthesis was calculated according to the following equation (5):

$$Inhibition rate cellular melanin synthesis ( \% ) = ( 1 - \frac{OD of a drug well - OD of a blank well}{OD of a control well - OD of the blank well} ) * 100 \% \qquad ( 5 )$$

TABLE 5
Results of inhibition rate of cellular melanin synthesis

| | | Inhibition rate of cellular |
|---|---|---|
| Samples | Concentration (g/mL) | melanin synthesis (%) |
| I-a | 0.10 | 71.10 |
| I-a | 0.050 | 50.22 |
| I-a | 0.025 | 18.55 |
| I-d | 0.10 | 67.80 |
| I-d | 0.050 | 50.40 |
| I-d | 0.025 | 33.54 |
| I-e | 0.10 | 34.99 |
| I-f | 0.05 | 48.15 |
| I-g | 0.05 | 43.26 |
| I-h | 0.10 | 46.30 |
| V-a | 0.010 | 73.80 |
| V-a | 0.0050 | 47.60 |
| V-a | 0.0025 | 28.89 |
| V-b | 0.10 | 32.05 |
| V-c | 0.10 | 54.85 |
| PC* | 0.30 | 66.37 |

*Note:
PC sample is kojic acid.

Test results show that compounds (I-a), (I-d), (I-e,) (I-f), (I-g), (I-h), (V-a), (V-b), and (V-c) of the embodiments of the present disclosure have an inhibitory effect on cellular melanin synthesis at a lower concentration (no more than 0.10% g/m L), and that compounds (I-a), (I-d), and (V-a) all exhibit an inhibitory effect on cellular melanin synthesis superior to that of kojic acid and are capable of inhibiting cellular melanin synthesis at a very low concentration.

The above results indicate that the compounds of the embodiments of the present disclosure all have strong inhibitory effect on cellular melanin and can be used alone or in combination as tyrosinase inhibitors for preparing whitening cosmetics, and for preventing and treating human pigmentation disorders caused by excessive melanin, melanoma and other diseases that require suppression of tyrosinase activity.

The foregoing is only a part of the embodiments of the present disclosure, and is not intended to limit the present disclosure, and any modifications, equivalent substitutions, improvements, etc., made within the spirit and principles of the present disclosure shall be included in the scope of protection of the present disclosure.

What is claimed is:

1. A compound, which is represented by a formula (A), 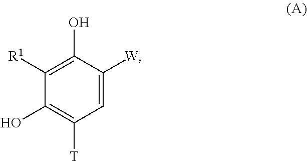
wherein W is 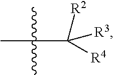
and T is H, 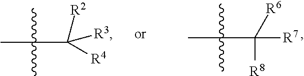
wherein R¹ is selected from H, an optionally substituted alkyl group, and an optionally substituted arylalkyl group;
R² and R⁶ are the same or different, which are independently selected from an optionally substituted aryl group, an optionally substituted heteroaryl group, the optionally substituted alkyl group, an optionally substituted amide group, and an optionally substituted ester group;
R³, R⁴, R⁷, and R⁸ are the same or different, which are independently selected from H, the optionally substituted alkyl group, an optionally substituted cycloalkyl group, and the optionally substituted amide group, wherein R³, R⁴, and a carbon atom to which R³ and R⁴ are both connected are connected to form a ring, R⁷, R⁸, and a carbon atom to which R⁷ and R⁸ are commonly linked are connected to form a ring, wherein the optionally substituted amide group includes at least one of —NHCOCH₃, —NHCOH, —NHCOCH₂CH₃, —NHCOCH₂CH₂CH₃, —NHCOCH(CH₃)₂, —NHCOCH(CH₂)₂, —N(COCH₃)₂, —CONH₂, —CON(CH₃)₂, —CONHCH₃, —CONHCH₂CH₃, —CON(CH₂CH₃)₂, —CONHCH(CH₃)₂, —CONHCH₂CH₂CH₃, —CONHCH₂CH₂CH₂CH₃, a phthalimido group, a succinimidyl group, a glutarimido group, a maleimido group, 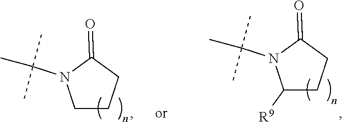
wherein R⁹ is selected from the optionally substituted alkyl group, the optionally substituted aryl group, a carboxyl or a salt thereof, a cyano group, the optionally substituted amide group, and the optionally substituted ester group, n is a natural number from 1 to 6, the optionally substituted ester group is —COOCH₃, —COOCH₂CH₃, —COOCH(CH₃)₂, —COOCH₂CH₂CH₃, or —COOCH₂CH₂CH₂CH₃;
or a cosmetically acceptable salt thereof or a pharmaceutically acceptable salt thereof, stereoisomers or mixtures of the stereoisomers, enantiomers or mixtures of the enantiomers, or racemic mixtures.

2. The compound of claim 1, wherein the compound is a compound represented by a formula (I)

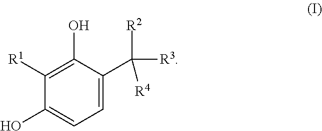

3. The compound of claim 1, wherein the compound is a compound represented by a formula (Ia), 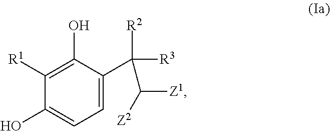
wherein
Z¹ and Z² are the same or different, which are independently selected from H, the optionally substituted alkyl group, the optionally substituted cycloalkyl group, and the optionally substituted amide group, wherein Z¹, Z², and a carbon atom to which Z¹ and Z² are commonly connected are connected to form a ring, and the ring is substituted.

4. The compound of claim 1, wherein the compound is a compound represented by a formula (V),

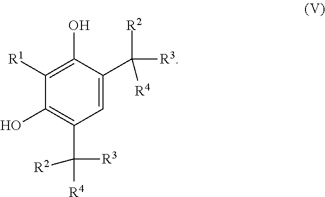

5. The compound of claim 4, wherein the compound is a compound represented by a formula (Va), 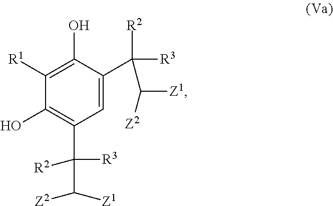
wherein
Z¹ and Z² are the same or different, which are independently selected from H, the optionally substituted alkyl group, the optionally substituted cycloalkyl group, and the optionally substituted amide group, wherein the Z¹, Z², and a carbon atom to which Z¹ and Z² are commonly connected are connected to form a ring, and the ring is substituted.

6. The compound of claim 1, wherein the compound is a compound represented by a formula (VI),

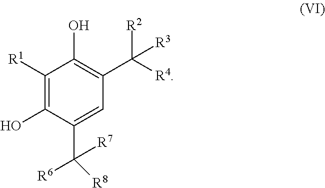

7. The compound of claim 6, wherein the compound is a compound represented by a formula (VIa), 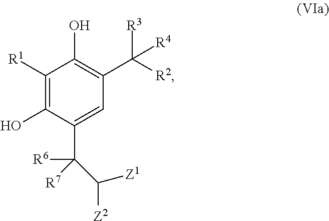
wherein
Z¹ and Z² are the same or different, which are independently selected from H, the optionally substituted alkyl group, the optionally substituted cycloalkyl group, and the optionally substituted amide group, wherein Z¹, Z², and a carbon atom to which Z¹ and Z² are commonly connected are connected to form a ring, and the ring is substituted.

8. The compound of claim 1, wherein the compound is selected from following compounds:

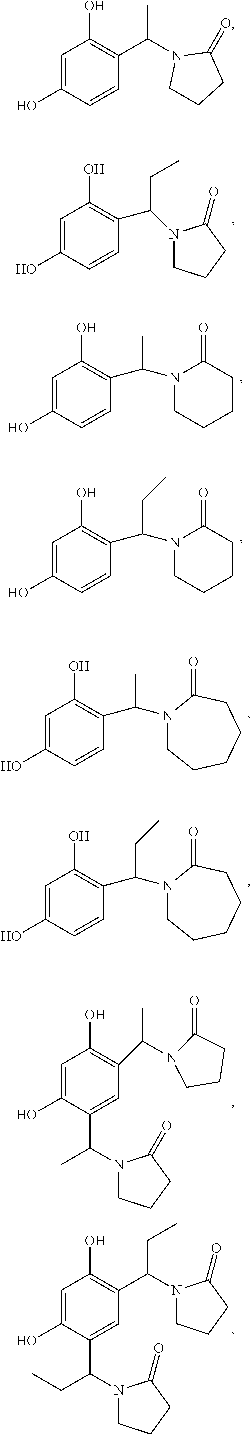

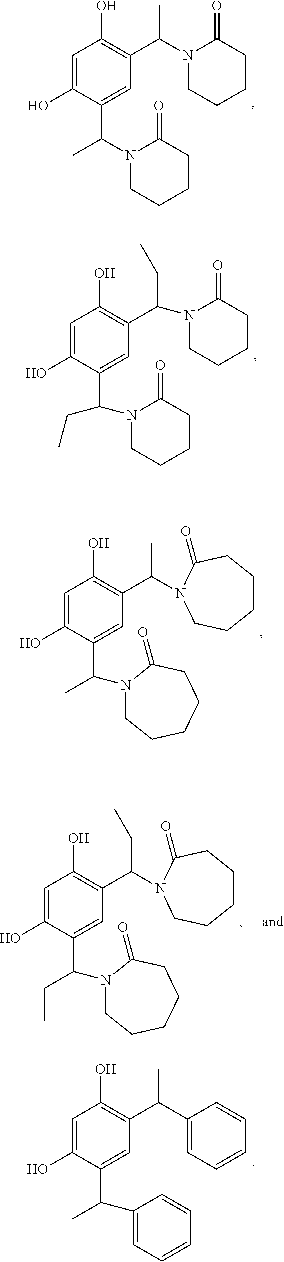

9. The compound of claim 2, wherein the compound represented by the formula (I) is prepared by reacting a compound represented by a formula (II) with a compound represented by a formula (IVa), and a preparation route (S1) is as follows: 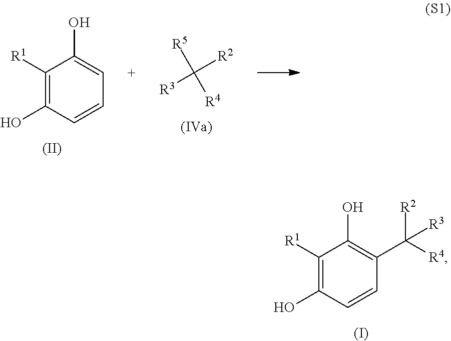
wherein R⁵ is selected from a leaving group, and the leaving group is at least one of OH, H₂O, OTs, OMs, Cl, Br, and I.

10. The compound of claim 3, wherein the compound represented by the formula (Ia) is prepared by reacting a compound represented by a formula (II) with a compound represented by a formula (IIIa), and a preparation route (S2) is as follows:

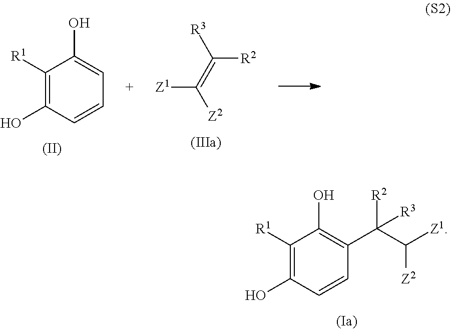

11. The compound of claim 4, wherein the compound represented by the formula (V) is prepared by reacting a compound represented by a formula (II) with a compound represented by a formula (IVa), and a preparation route (S3) is as follows: 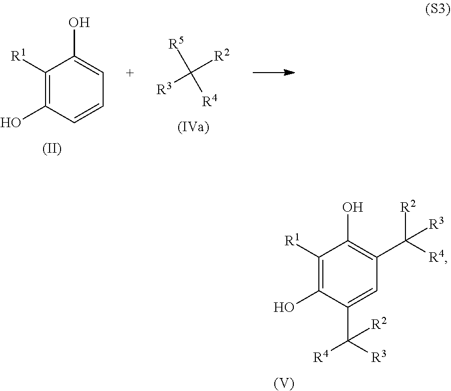
wherein the R⁵ is selected from a leaving group, and the leaving group is at least one of OH, H₂O, OTs, OMs, Cl, Br, and I.

12. The compound of claim 5, wherein the compound represented by the formula (Va) is prepared by reacting a compound represented by a formula (II) with a compound represented by a formula (IIIa), and a preparation route (S4) is as follows:

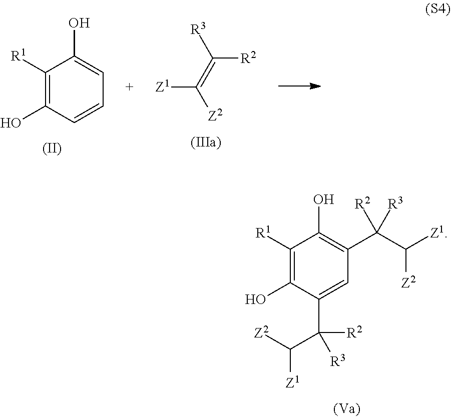

13. The compound of claim 6, wherein the compound represented by the formula (VI) is prepared by reacting a compound represented by a formula (I) with a compound represented by a formula (IVb), and a preparation route (S5) is as follows: 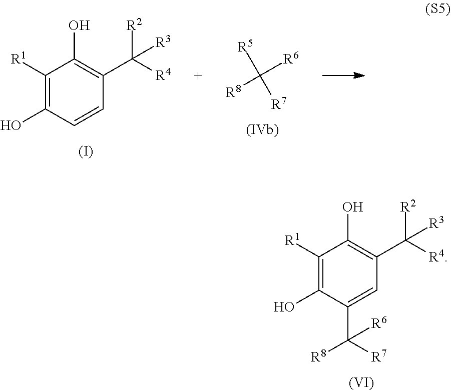
wherein the R⁵ is selected from a leaving group, and the leaving group is at least one of OH, H₂O, OTs, OMs, CI, Br, and I.

14. The compound of claim 7, wherein the compound represented by the formula (VIa) is prepared by reacting a compound represented by a formula (I) with a compound represented by a formula (IIIb), and a preparation route (S6) is as follows:

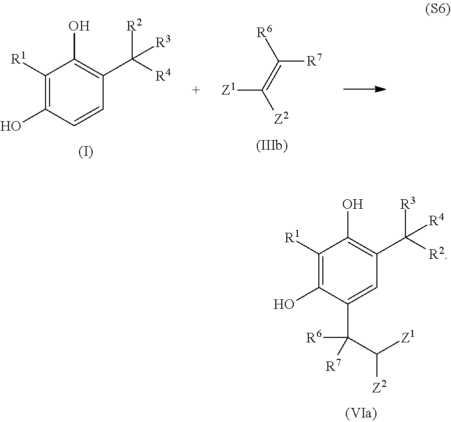

15. A pharmaceutical composition, comprising the compound of claim 1 or a pharmaceutically acceptable salt thereof.

16. A cosmetic composition, comprising the compound of claim 1 or a pharmaceutically acceptable salt thereof.
